(19) United States
(12) Patent Application Publication
(10) Pub. No.: US 2025/0213654 A1
(43) Pub. Date: Jul. 3, 2025

(54) PEPTIDES AND METHODS FOR USE IN TREATING PAIN

(71) Applicant: TAFALGIE THERAPEUTICS, MARSEILLE (FR)

(72) Inventors: ABDELAZIZ MOQRICH, MARSEILLE (FR); STÉPHANE GAILLARD, MARSEILLE (FR)

(21) Appl. No.: 18/868,199

(22) PCT Filed: May 25, 2023
(86) PCT No.: PCT/EP2023/064091

§ 371 Date: Nov. 22, 2024

(30) Foreign Application Priority Data
May 25, 2022 (EP) 22305773.8

(51) Int. Cl. A61K 38/19; A61K 9/00; A61K 45/06; A61K 47/12; A61P 25/04

(52) U.S. Cl. CPC A61K 38/195; A61K 9/0019; A61K 9/0053; A61K 45/06; A61K 47/12; A61P 25/04

(57) ABSTRACT

The present invention relates to a set of peptides, to compositions and kits comprising or allowing the expression of said peptides, as well as to uses thereof, in particular for preventing or treating pain in a subject in need thereof.

### FIELD OF THE INVENTION

The present invention relates generally to the field of pain management. More particularly, the invention relates to a set of peptides, to compositions and kits comprising, or allowing the expression of, said peptides, as well as to uses thereof, in particular for preventing or treating pain in a subject in need thereof. The peptides of the set are specific fragments of the human TAFA-4 protein, or equivalent fragments thereof, usable as active ingredients for preventing or treating pain, in particular acute, subacute, or chronic pain originating from neuropathic pain, post-operative pain, or inflammatory pain, or for preventing or treating hyperalgesia or allodynia resulting from injury-induced pain.

### BACKGROUND OF THE INVENTION

Pain is commonly classified as acute or chronic. Acute pain is short-lived and essential for the maintenance of our physical integrity, whereas chronic pain persists beyond the normal time of healing and adversely affects well-being. Chronic inflammatory, neuropathic or postoperative pains give rise to long-lasting sensory abnormalities such as hyperalgesia (extreme pain evoked by noxious stimuli) and mechanical allodynia (pain elicited by innocuous mechanical stimuli). These categories of pain differ in terms of etiology and clinical features, but they have several mechanisms in common, including alterations to neuro-immune interactions and neuron sensitization, both peripherally and centrally (Costigan et al., 2009). There is growing evidence to suggest that loss of inhibition may be a key mechanism underlying chronic pain (Bourane et al., 2015a; Bourane et al., 2015b; Boyle et al., 2019; Coull et al., 2005; Duan et al., 2014; Peirs et al., 2015; Petitjean et al., 2019; Petitjean et al., 2015; Zeilhofer et al., 2012; Zhang et al., 2018, Yoo et al., 2021). However, despite our extensive knowledge of the mechanisms and circuits underlying chronic pain in rodents, the translation of these findings into effective treatments for chronic pain in humans remains unsatisfactory (Colloca et al., 2017). Indeed, non-steroidal anti-inflammatory agents (NSAIDs) have limited efficacy against chronic pain, and opioids have multiple adverse effects, including potentially lethal respiratory depression, nausea, constipation, hyperalgesia, tolerance, physical and psychological dependence (Benyamin et al., 2008). Thus, efforts to identify new targets with analgesic or antalgic potential for the treatment of chronic pain should be encouraged.

In the last few years, inventors have discovered striking features of the secreted protein TAFA-4 suggesting that it might be an interesting drug for the treatment of chronic pain (WO2014180853).

TAFA-4 belongs to a family of five highly conserved secreted neurokines (Sarver et al., 2021). TAFA-4 contains a signal peptide followed by a highly conserved core region with 10 cysteine residues including a CC-chemokine motif that makes it resemble a cytokine (Tom Tang et al., 2004).

Inventors have also previously shown that in mice in which the TAFA-4 gene is deleted, mechanical hypersensitivity induced by nerve injury lasts much longer than in wild-type (WT) mice (Delfini et al., 2013). These phenotypes are reversed by intrathecal injection of recombinant TAFA-4.

As described in WO2014180853, inventors previously reported experimental evidence demonstrating that TAFA-4 possesses powerful actions against pathologically increased mechanical pain. It has been shown that, in both male and female mice, the intrathecal and subcutaneous administration of human recombinant TAFA-4 reverses inflammatory, postoperative and nerve injury-induced mechanical hypersensitivity. They have also shown that TAFA-4 can reverse nerve injury-induced neuronal sensitization of the spinal lamina II interneurons reported to be responsible for mechanical threshold alterations. Production of recombinant mature TAFA-4 protein may, however, be challenging because TAFA-4 is a cysteine-rich protein (10 cysteines in total). Indeed, refolding can result in obtaining non-native conformations or an improper disulfide bridging pattern which may greatly impact the activity of the protein. Moreover, the several purification steps required for obtaining a recombinant TAFA-4 protein with high degree of purity, together with possible aggregation problems, may limit the yield of a properly folded protein. Furthermore, while chemical synthesis of TAFA-4 protein was implemented, it is also challenging due to the length of the protein, requiring assembly of not less than 4 (protected or partially protected) peptide segments (starting from the N terminal and ending with the most C-terminal peptide). To generate a full length TAFA-4, three ligation purification steps are required, leading to low yields that are not compatible with industrial scale production. Thus, there is a strong need for alternative therapeutic agents and methods for use for efficiently preventing or treating pain, in particular acute, subacute or chronic pain, and there would be substantial advantage in identifying novel compounds having a similar or better activity than that of the full length TAFA-4 protein, that can be manufactured on an industrial scale and in a cost-effective way.

### SUMMARY OF THE INVENTION

The present invention is based, at least in part, on the identification of peptides exhibiting an antalgic or analgesic activity, in particular an antalgic activity. Inventors have discovered that these peptides are advantageously able to reverse the mechanical-hypersensitivity induced by nerve damage, surgery or by the injection of an inflammatory agent such as carrageenan, i.e. are capable of treating a neuropathic, postoperative or inflammatory pain.

The peptides of interest are, or derive from, specific fragments of the human TAFA-4 protein. They are more efficacious against pain, in particular chronic pain, typically against injury-induced pain than the human TAFA-4 full length protein (herein also identified as “TAFA-4 FL”), and are easier to produce. Indeed, these peptides are short peptides which do not need to be linked to each other to generate a therapeutic effect. Inventors herein provide patients with a new tool capable of alleviating the problems of addiction to pain treatment.

Inventors in particular herein describe for the first time a set of products or components, in particular a set of peptides, of nucleic acids encoding such peptides, of vectors and/or of cells allowing the expression of such peptides, the peptides comprising, or consisting of, a first peptide of SEQ ID NO: 1 (CFPGQVAGTTRAQPSCVEASIVIQKWW) or a peptide having at least 90% identity to SEQ ID NO: 1 (also herein identified as “variant”, “peptide variant” or “variant of the peptide” of sequence SEQ ID NO: 1), and a second peptide of SEQ ID NO: 2 (CHMNPCLEGEDCKVLPDYSGWSCSSGNKVKTTKVTR) or a peptide having at least 90% identity to SEQ ID NO: 2 (also herein identified as “variant”, “peptide variant” or “variant of the peptide” of sequence SEQ ID NO: 2), wherein said (at least two) peptides are not linked to each other. The variant is typically a functional variant, i.e. a variant having the same (therapeutic) effect as that herein described by inventors for the peptide of SEQ ID NO: 1 or SEQ ID NO: 2.

Also herein described is a composition comprising a peptide of sequence SEQ ID NO: 1 or a variant thereof (i.e., a peptide having at least 90% identity to SEQ ID NO: 1), and a peptide of sequence SEQ ID NO: 2 or a variant thereof (i.e., a peptide having at least 90% identity to SEQ ID NO: 2), wherein said (at least two) peptides are not linked to each other.

In a particular aspect, similarly to the peptides of SEQ ID NO: 1 and of SEQ ID NO: 2, their herein described variants modulate excitability of spinal cord interneurons (preferably spinal cord lamina IIi interneurons).

In a preferred aspect, the glutamine (Q) residue at position 13 in the peptide of SEQ ID NO: 1 (/with reference to position set forth in SEQ ID NO: 1) remains unchanged in the variant of the peptide of SEQ ID NO: 1. Similarly, the tyrosine (Y) residue at position 18 in the peptide of SEQ ID NO: 2 (/with reference to position set forth in SEQ ID NO: 2), either remains unchanged in the variant of the peptide of SEQ ID NO: 2 or is replaced by a serine (S).

Inventors herein describe in particular a set of nucleic acids allowing the expression of (at least two) peptides which are not linked to each other, wherein a (first) nucleic acid comprises a sequence encoding a peptide of SEQ ID NO: 1 or a peptide having at least 90% identity to SEQ ID NO: 1, and a (second) nucleic acid comprises a sequence encoding a peptide of SEQ ID NO: 2, or a peptide having at least 90% identity to SEQ ID NO: 2, as well as a composition comprising such a set of nucleic acids.

Also herein disclosed is a set of vectors allowing the expression of (at least two peptides) which are not linked to each other, wherein a (first) vector comprises a sequence encoding a peptide of SEQ ID NO: 1 or a peptide having at least 90% identity to SEQ ID NO: 1, and a (second) vector comprises a sequence encoding a peptide of SEQ ID NO: 2, or a peptide having at least 90% identity to SEQ ID NO: 2, as well as a composition comprising such a set of vectors.

Further herein disclosed is a cell allowing the expression of (at least two) peptides (or variants thereof) as herein described which are not linked to each other, and wherein i) the cell comprises a (first) nucleic acid sequence or vector encoding a peptide of SEQ ID NO: 1 or a peptide having at least 90% identity to SEQ ID NO: 1, and a (second) nucleic acid sequence or vector encoding a peptide of SEQ ID NO: 2 or a peptide having at least 90% identity to SEQ ID NO: 2. Also disclosed is a composition comprising such a cell and an acceptable support.

Also herein described is a set, composition or cell as herein disclosed for the first time for use as an active therapeutic or prophylactic ingredient, typically for use as a pharmaceutical composition, drug or medicament.

Any of the herein described compositions is typically a pharmaceutical, therapeutic, veterinary or dietetically/dietarily composition comprising in addition to the (at least two) peptides a pharmaceutically and/or dietetically/dietarily acceptable support.

In a particular aspect, the composition may further comprise at least one additional (distinct) active compound/agent, preferably an active agent efficient against pain (acute, subacute or chronic pain), even more preferably a steroidal anti-inflammatory drug (SAID), a non-steroidal anti-inflammatory drug (NSAID) or an opioid drug.

The present invention also relates to a set, or cell as herein described, typically to a set of peptides, a set of nucleic acids encoding such peptides, or a set of vectors allowing the expression of such peptides, or to a cell comprising or expressing such a set, for use [as (an) active(s) ingredient(s)] for preventing or treating pain in a subject in need thereof. It also relates to the use of such peptides or such a set for the manufacture of a pharmaceutical composition, drug or medicament for preventing or treating pain in a subject in need thereof. In particular, the invention may be used to treat a chronic pain, a neuropathic pain, a postoperative pain, an inflammatory pain, hyperalgesia, allodynia or acute pain. The invention may also be used to treat an arthritic pain, pain associated with damage to joints, pain related to Ehlers-Danlos syndrome, pain resulting from sun burn or pain resulting from ice burn.

The present invention also relates to a method of preventing or treating pain in a subject in need thereof, comprising administering a (therapeutic effective) amount of a product such as a set and/or composition as herein described, preferably a set of peptides comprising the peptide of SEQ ID NO: 1, or a variant thereof having at least 90% identity to SEQ ID NO: 1, and the peptide of SEQ ID NO: 2, or a variant thereof having at least 90% identity to SEQ ID NO: 2, or composition comprising such a set.

The products, in particular the peptides, sets and compositions, as herein described may be administered to the subject by any route, such as for example intramuscularly, intravenously, intraperitoneally, orally (per os), anally, cutaneously, subcutaneously, topically, dermically, transdermically or intrathecally. Preferably, the product herein described, for example the peptides or the composition, is administered to the subject subcutaneously, orally or intravenously, even more preferably subcutaneously or orally.

Each of the products, for example the peptides, or of the compositions described herein may be part of a kit.

A particular kit of the invention comprises i) a first peptide of SEQ ID NO: 1, a peptide having at least 90% identity to SEQ ID NO: 1, or a nucleic acid sequence encoding such a peptide, ii) a second peptide of SEQ ID NO: 2, a peptide having at least 90% identity to SEQ ID NO: 2, or a nucleic acid sequence encoding such a peptide, the first and second peptides, or the nucleic acids encoding said peptides, the first and second peptides, or the nucleic acids encoding said peptides, being in distinct containers, and optionally iii) written instructions for using the kit. The kit herein described is typically for use in the context of the prevention or treatment of pain.

In a particular aspect, the products of the present invention may also be used in the context of research. The nucleic acids encoding a peptide or a variant thereof as herein described, the vectors permitting their expression, or the cell comprising such nucleic acid(s) or modified using such vector(s) may be used to express or modulate in vitro or ex vivo the expression of a peptide of SEQ ID NO: 1 or a functional variant thereof having at least 90% identity to SEQ ID NO: 1 and/or of a peptide of SEQ ID NO: 2 or a functional variant thereof having at least 90% identity to SEQ ID NO: 2. In another particular aspect, the present invention also relates to a transgenic animal modified to express at least two distinct herein described nucleic acids.

### BRIEF DESCRIPTION OF THE FIGURES

FIG. 1: Comparison of the antalgic effects of TAFA-4 (full length protein) and of the set of two peptides of SEQ ID NO: 1 and of SEQ ID NO: 2, when administered subcutaneously in vivo in a Spared Nerve Injury (SNI) model of neuropathic pain. Mice suffering of mechanical hypersensitivity are subcutaneously administered with the set of two peptides of SEQ ID NO: 1 and of SEQ ID NO: 2 (this set being herein identified as “Peptides 1+2”), the TAFA-4 full length protein (identified as “TAFA4 FL”), a negative control (vehicle) or a positive control (Pregabalin). The figure illustrates the mechanical force required (expressed as its weight equivalent in grams) to elicit a paw withdrawal response in 50% of mice as a function of time under each condition. Data are represented as percent response relative to baseline levels. The measures were performed before implementation of the SNI pain model (T0=baseline), 14 days after SNI to validate establishment of the mechanical allodynia, and then at 1, 2 and 4 hours post subcutaneous injection. Statistical differences between “Peptides 1+2”-treated mice, “TAFA-4 FL”-treated mice, and vehicle groups are shown (Two-way RM ANOVA, followed by Bonferroni post-hoc test: *p<0.05, **p<0.01, ***p<0.001).

FIG. 2: Comparison of the antalgic effects of TAFA-4 (full length protein) and of the set of two peptides of SEQ ID NO: land of SEQ ID NO: 2, when administered per os in vivo ha SNI model of neuropathic pain. Mice suffering of mechanical hypersensitivity are administered per os with the set of two peptides of SEQ ID NO: 1 and of SEQ ID NO: 2 (this set being herein identified as “Peptides 1+2”), the TAFA-4 full length protein (identified as “TAFA4 FL”), a negative control (vehicle) or a positive control (Pregabalin). The figure illustrates the mechanical force required (expressed as its weight equivalent in grams) to elicit a paw withdrawal response in 50% of mice as a function of time under each condition. Data are represented as percent response relative to baseline levels. The measures were performed before implementation of the SNI pain model (T0=baseline), 14 days after SNI to validate establishment of the mechanical allodynia, and then at 1, 2 and 4 hours post oral gavage. Statistical differences between “Peptides 1+2”-treated mice, “TAFA-4 FL”-treated mice, and vehicle groups are shown (Two-way RM ANOVA, followed by Bonferroni post-hoc test: *p<0.05, **p<0.01, ***p<0.001).

FIG. 3: Antalgic effect of the set of two peptides of SEQ ID NO: 1 and of SEQ ID NO: 2 when administered intravenously in vivo in a SNI model of neuropathic pain. Mice suffering of mechanical hypersensitivity are administered intravenously with the set of two peptides of SEQ ID NO: 1 and of SEQ ID NO: 2 (this set being herein identified as “Peptides 1+2”) or a negative control (vehicle). The figure illustrates the mechanical force required (expressed as its weight equivalent in grams) to elicit a paw withdrawal response in 50% of mice as a function of time under each condition. Data are represented as percent response relative to baseline levels. The measures were performed before implementation of the SNI pain model (T0=baseline), 14 days after SNI to validate establishment of the mechanical allodynia, and then at 1, 2 and 4 hours post intravenous injection. Statistical differences between “Peptides 1+2”-treated mice and the vehicle group are shown (Two-way RM ANOVA, followed by Bonferroni post-hoc test: *p<0.05, **p<0.01, ***p<0.001).

FIG. 4: Antalgic effect of the set of two peptides of SEQ ID NO:1 and of SEQ ID NO:2 when administered subcutaneously in vivo in a postoperative pain model (Brennan paw incision model) in both male and female mice.

A. Schematic representation of the protocol. The set of two peptides of SEQ ID NO:1 and of SEQ ID NO:2 or a negative control (vehicle) were administered subcutaneously twice a day at different time points: the day before surgery (D-1), the day of surgery (D0), and at the days D+1 and D+2 after surgery. The administrations at D0 were performed 1 hour before and 1 hour after surgery (and wake up), respectively. Mechanical threshold response measurements at D+1 and D+2 were performed before administration of the set of two peptides or vehicle.

B. The figure represents the mechanical force required (expressed in percentage of baseline levels) to elicit a paw withdrawal response in 50% of mice in function of time. The mice treated with the set of peptides of SEQ ID NO: 1 and SEQ ID NO:2 (this set being herein identified as “Peptides 1+2”) were compared to the mice treated with a negative control (vehicle). Statistical differences between “Peptides 1+2” treated mice and the vehicle group are shown (Two-way RM ANOVA, followed by Bonferroni post-hoc test: ***p<0.001). “Peptides 1+2”-treated group: 24 mice, vehicle group: 22 mice; Model=postoperative pain model; Sex: 12 males and 12 females for mice treated with “Peptides 1+2”, 11 males and 11 females for vehicle group; Administration: subcutaneously; Total concentration of “Peptides 1+2”: 300 μg/kg. (that corresponds approximatively for Peptide 1: 128 μg/kg and for Peptide 2: 172 μg/kg)

FIG. 5: Absence of tolerance after repeated subcutaneous administration of the set of peptides of SEQ ID NO:1 and of SEQ ID NO:2 on Chronic Constriction Injury (CCI) neuropathic pain model.

A. Schematic representation of the protocol. After establishment of CCI neuropathic pain model, mice were treated with the set of peptides of SEQ ID NO: 1 and of SEQ ID NO:2 (this set being herein identified as “Peptides 1+2”) or a negative control (vehicle) for twice a day during 14 consecutive days starting 7 days after surgery. Antalgic effect of “Peptides 1+2” was determined every two days.

B. The figure represents the mechanical force required (expressed in percentage of baseline levels) to elicit a paw withdrawal response in 50% of mice in function of time. The mice treated with “Peptide 1+2” were compared to mice treated with a negative control (vehicle). Statistical differences between antalgic effect after the first and the last administration of “Peptide 1+2”-treated mice and vehicle group am shown (Two-way RM ANOVA, followed by Bonferroni post-hoc test: ns: non-significant). Model=CCI neuropathic pain model; Sex: 10 males for “Peptides 1+2”-treated mice, n=9 for vehicle group; Administration: subcutaneously; Total concentration of “Peptides 1+2”: 300 μg/kg.

FIG. 6: Comparison of different posology of treatment with the set of peptides of SEQ ID NO:1 and of SEQ ID NO:2 when administered subcutaneously in vivo in a postoperative pain model (Brennan paw incision model) in both male and female mice.

A. Schematic representation of the protocol. The set of two peptides of SEQ ID NO:1 and of SEQ ID NO:2(“Peptides 1+2”) or a negative control (vehicle) were administered subcutaneously twice a day according to three different protocols. “D-1/D0” protocol: the day before surgery (D-1) and the day of surgery (D0); “D0” protocol: the day of surgery (D0); “D0/D+1/D+2” protocol: the day before surgery (D-1), the day of surgery (D0), and at the days D+1 and D+2 after surgery. The administrations at D0 were performed 1 hour before and 1 hour after surgery (and wake up), respectively. Mechanical threshold response measurements at D+1 and D+2 were performed before administration of the set of two peptides or vehicle.

B. The figure represents the mechanical force required (expressed in percentage of baseline levels) to elicit a paw withdrawal response in 50% of mice in function of time. The mice treated with “Peptides 1+2” were compared to the mice treated with a negative control (vehicle) according to the three different protocols above described “D-1/D0”, “D0”, “D0/D+1/D+2”. Statistical differences between “Peptides 1+2”-treated mice and vehicle group are shown (Two-way RM ANOVA, followed by Bonferroni post-hoc test: **p<0.01, ***p<0.001). “Peptides 1+2”-treated group: 16 mice; vehicle group: 11 mice; Model=postoperative pain model; Sex: 8 males and 8 females for mice treated with “peptides 1+2”, 6 males and 5 females for vehicle group; Administration: subcutaneously; Total concentration of “Peptides 1+2”: 300 μg/kg.

FIG. 7: Antalgic effect of the set of peptides of SEQ ID NO: 1 and of SEQ ID NO:2 administered subcutaneously in vivo in an inflammatory pain model (capsaicin inflammatory pain model). The figure represents the mechanical force required (expressed in percentage of baseline levels) to elicit a paw withdrawal response in 50% of mice in function of time. The mice treated with a set of peptides of SEQ ID NO:1 and of SEQ ID NO:2 (this set being herein identified as “Peptides 1+2”) were compared to mice treated with a negative control (vehicle). Statistical differences between “Peptides 1+2”-treated mice and vehicle group are shown (Two-way RM ANOVA, followed by Bonferroni post-hoc test: *p<0.05, **p<0.01, ***p<0.001). Model=inflammatory pain model; “Peptides 1+2”-treated group: 8 male mice vehicle group: 8 male mice; Administration: subcutaneously; Total concentration of “Peptides 1+2”: 300 μg/kg.

FIG. 8: Antalgic effect of the set of peptides of SEQ ID NO: 1 and of SEQ ID NO:2 administered subcutaneously in vivo in osteoarthritis pain model (monoiodoacetate model).

The figure represents the mechanical force required (expressed in percentage of baseline levels) to elicit a paw withdrawal response in 50% of mice in function of time. The mice treated with a set of peptides of SEQ ID NO:1 and of SEQ ID NO:2 (this set being herein identified as “Peptides 1+2”) were compared to the mice treated with a negative control (vehicle). Statistical differences between “Peptides 1+2”-treated mice and vehicle group are shown (Two-way RM ANOVA, followed by Bonferroni post-hoc test: *p<0.05, **p<0.01, ***p<0.001). Model=osteoarthritis pain model; “Peptides 1+2”-treated group: 8 male mice, vehicle group: 8 male mice; Administration: subcutaneously; Total concentration of “Peptides 1+2”: 300 μg/kg.

FIG. 9: Antalgic effect of the set of peptides of SEQ ID NO: 1 and of SEQ ID NO: 2 administered subcutaneously in vivo in Freeze-burn pain model.

The figure represents the mechanical force required (expressed in percentage of baseline levels) to elicit a paw withdrawal response in 50% of mice in function of time. The mice treated with a set of peptides of SEQ ID NO:1 and of SEQ ID NO:2 (this set being herein identified as “Peptides 1+2”) were compared to the mice treated with a negative control (vehicle). Statistical differences between “Peptides 1+2”-treated mice and vehicle group are shown (Two-way RM ANOVA, followed by Bonferroni post-hoc test: *p<0.05, **p<0.01, ***p<0.001). Model=freeze-burn pain model; “Peptides 1+2”-treated group: 8 male mice, vehicle group: 8 male mice; Administration: subcutaneously; Total concentration of “Peptides 1+2”: 300 μg/kg.

FIG. 10: Antalgic effect of the set of peptides of SEQ ID NO: 1 and of SEQ ID NO:2 administered subcutaneously in vivo in Ultraviolet-burn pain model.

The figure represents the mechanical force required (expressed in its equivalent in weight in gram) to elicit a paw withdrawal response in 50% of mice in function of time. The data are represented as percentage response to baseline levels. The mice treated with a set of peptides of SEQ ID NO:1 and of SEQ ID NO:2 (this set being herein identified as “Peptides 1+2”) were compared to the mice treated with a negative control (vehicle). Statistical differences between “Peptides 1+2”-treated mice and vehicle group are shown (Two-way RM ANOVA, followed by Bonferroni post-hoc test: *p<0.05, **p<0.01, ***p<0.001). Model=ultraviolet-burn pain model; 16 mice (8 male and 8 female mice) for each group; Administration: subcutaneously; Total concentration of “Peptides 1+2”: 300 μg/kg.

### DETAILED DESCRIPTION OF THE INVENTION

### Definitions

The terms used in this specification generally have their ordinary meanings in the art within the context of this invention and the specific context where each term is used. Certain terms are discussed below, or elsewhere in the specification, to provide additional guidance to the skilled reader in describing the methods of the invention and how to use them. Moreover, it will be appreciated that the same item can be said in more than one way. Consequently, alternative language and synonyms may be used for any one or more of the terms discussed herein. Synonyms for certain terms are provided. A recital of one or more synonyms does not exclude the use of the other synonyms.

The terms “peptide”, “peptide variant”, “protein fragment”, “protein segment” are used interchangeably to refer to a sequence or polymer of amino acid residues. Such polymers of amino acid residues may contain natural or non-natural amino acid residues. The terms also include post-expression modifications of the peptide, for example, glycosylation, sialylation, acetylation, amidation, phosphorylation, carbamethylation and the like. Furthermore, in the context of the present invention, a “peptide” refers to a protein fragment or segment which may include such a post-expression modification or a combination of such modifications, optionally together with one or several modifications such as deletions, additions, and substitutions (generally conservative in nature), to the native (wild-type) sequence, as long as the peptide maintains the desired activity, i.e. preventing or treating pain. These modifications are preferably deliberate mutations, e.g. are obtained through site-directed mutagenesis.

The terms “isolated peptide” refers to a peptide that is removed from its original environment (i.e. the natural environment, if it is naturally occurring). A peptide naturally present in a natural system, for example in a living animal, is to be distinguished from the same peptide, which has been separated from all or part of the coexisting materials in said natural system. The separated peptide is herein designated as an “isolated peptide”. In other words, in the context of the present invention the isolated peptide is a fragment of the TAFA-4 protein which is absent as such from nature.

The terms “synthetic peptide” refer to a peptide obtained by chemical synthesis.

The terms “set of peptides” or “peptide set”, as used herein, refer to at least two peptides which are used in combination as active ingredients (of the set), possibly mixed in a composition, but which are not linked to each other. If there are two peptides, the peptides of the set are not bound together (if the peptides of the set are more than two, these peptides are not bound to each other) by any bond, for example by a bond/link consisting of a peptide (sequence of amino acids), or by a bond/link of a chemical nature or of both organic and chemical nature. The peptides of the set are isolated peptides, synthetic peptides or a mix thereof. The set may be in the form of a dry powder, solution, suspension or colloid, and may be homogenous or heterogeneous. The set of peptides is generally part of a composition. The composition typically also comprises a pharmaceutically and/or dietetically acceptable support, carrier, excipient or vehicle. Typically, the excipient can be ascorbic acid and/or an ascorbic acid salt. Ascorbic acid or L-acid ascorbic (IUPAC name:(5R)-5-[(1S)-1,2-dihydroxyethyl]-3,4-dihydroxyfuran-2(5H)-one) is a naturally-occurring water-soluble vitamin commonly known as Vitamin C. Examples of ascorbic acid salts include, without limitation, Magnesium Ascorbate, Sodium Ascorbate, Calcium Ascorbate and Ascorbyl Palmitate. L-ascorbic acid and its sodium salt are antioxidants and are useful as preservatives or stabilizers when used in combination with the set of peptides. Other antioxidants (for ex, methionine) could also be used.

The terms “sequence identity”, “sequence having at least X % identity” and “sequence X % identical to”, X being for example 90, are used interchangeably to refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Preferably, sequence identity is determined over the entire length of a reference sequence, here SEQ ID NO: 1 or SEQ ID NO: 2.

Optimal alignment of sequences for comparison can be conducted, e.g., by the local homology algorithm of Smith and Waterman, (1981) Adv. Appl. Math. 2:482, by the homology alignment algorithm of Needleman and Wunsch, (1970) J. Mol. Biol. 48:443, by the search for similarity method of Pearson and Lipman, (1988) Proc. Natl. Acad. Sci. USA 85:2444, by computerized implementations of these algorithms (GAP, BESTFIT, FASTA, and TFASTA), or by visual inspection (see generally, Current Protocols in Molecular Biology, F. M. Ausubel et al., eds., Current Protocols, Greene Publishing Associates, Inc. and John Wiley & Sons, Inc., (1995 Supplement)).

Examples of algorithms that are suitable for determining percent sequence identity and sequence similarity are the BLAST and BLAST 2.0 algorithms, which are described in Altschul et al., (1990), J. Mol. Biol. 215: 403-410 and Altschul et al., (1977) Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T” when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (“E”) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, (1989) Proc. Natl. Acad. Sci. USA 89:10915).

The degree of percent amino acid sequence identity can also be obtained by ClustalW analysis (version W 1.8) by counting the number of identical matches in the alignment and dividing such number of identical matches by the length of the reference sequence, and using the following default ClustalW parameters to achieve slow/accurate pairwise optimal alignments—Gap Open Penalty: 10; Gap Extension Penalty: 0.10; Protein weight matrix: Gonnet series; DNA weight matrix: IUB; Toggle Slow/Fast pairwise alignments=SLOW or FULL Alignment.

The terms “nucleic acid” or “polynucleotide” as used herein refer to a polymeric form of nucleotides of any length, either ribonucleotides or deoxyribonucleotides. Thus, these terms include, but are not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “expression cassette”, “nucleic acid construction” or “nucleic acid construct” are herein used interchangeably and refer to a nucleic acid comprising at least one transcriptional promoter operably linked (with the meaning understood by persons skilled in the art) to one or more (coding) sequences of interest, typically to an operon comprising several coding sequences of interest whose expression products contribute to the creation of a function of interest within a cell, or to a nucleic acid further comprising an activation sequence and/or transcription terminator.

The term “vector” refers i) to a DNA or RNA molecule that comprises a polynucleotide sequence, for example an expression cassette or a transgene, that encodes a peptide, a polypeptide or a protein, or ii) to a product encapsulating such a DNA or RNA molecule. A vector generally contains regulatory elements capable of directing expression of the encoding polynucleotide sequence, also called transgene, in the cells into which the nucleic acid molecule is introduced. The term “transgene” refers to a polynucleotide that is introduced into a cell and is capable of being transcribed into RNA and optionally, translated and/or expressed under appropriate conditions. In certain aspects, it confers a desired property to a cell into which it was introduced, or otherwise leads to a desired technical effect, here typically a therapeutic effect. A transgene may contain sequence(s) coding for one or more proteins or one or more fragments of proteins. In a particular aspect, the vector is a plasmid. Alternatively, the vector is any well-known vehicle capable of artificially carrying a foreign nucleic acid sequence of interest as herein described, such as for example a viral vector, a cosmid, a phage or a double lipid membrane (such as a liposome) encapsulating a product of interest as herein described usable to deliver it on a site of interest, etc.

The terms “gene therapy” refer to the treatment of a subject which involves delivery of a gene/nucleic acid into the cells of an individual for the purpose of preventing or treating a disease.

The term“transfection” refers to the uptake of a foreign polynucleotide by a cell, such as a prokaryote or eukaryote cell. A cell is identified as “transfected” when exogenous polynucleotide has been introduced into the cell. A number of transfection techniques are generally known in the art. See, e.g., Graham et al., Virology 52:456 (1973), Sambrook et al, Molecular Cloning, a Laboratory Manual, Cold Spring Harbor Laboratories, New York (1989), Davis et al., Basic Methods in Molecular Biology, Elsevier (1986), and Chu et al., Gene 13:197 (1981). Such techniques can be used to introduce one or more exogenous nucleic acids into suitable host cells.

The term “transduction” refers to the delivery of a nucleic acid molecule into a recipient host cell, such as by a gene delivery vector, for example a recombinant viral vector, in particular a retrovirus, an adenovirus, a recombinant adeno-associated virus (AAV), a herpes simplex virus or a lentivirus. For example, transduction of a target cell by a rAAV virion leads to transfer of the rAAV vector contained in that virion into the transduced cell.

A recombinant “adeno-associated virus (AAV)” is a small Dependoparvovirus with a single-stranded linear DNA genome, lacking pathogenicity and presenting low immunogenicity, that has been artificially produced using recombinant methods. Recombinant AAVs (rAAVs) preferably have tissue/cell-specific targeting capabilities, such that a transgene of the rAAV will be delivered specifically or preferentially to one or more predetermined tissue(s)/cell(s). The AAV capsid as well as the type of regulatory region and administration route are important elements in determining these tissue-specific targeting capabilities.

The term “pain”, refers within the context of the present invention to any pain or sensitivity associated with tissue damage. Preferably, the term pain as used therein is understood as an abnormal sensitivity, i.e., typically as a hypersensitivity which is mediated by aberrantly increased activity of all types of sensory neurons including nociceptors and non-nociceptors. The term pain includes any pain selected from a nociceptor-mediated pain (also called herein a “nociceptive pain”), a neuropathic pain, an inflammatory pain, a pathological pain, an acute pain, a subacute pain, a chronic pain, a mechanical pain, a chemical pain, a somatic pain, a visceral pain, a deep somatic pain, a superficial somatic pain, a somatoform pain, allodynia, hyperalgesia, or a pain associated with a nerve injury.

“Nociceptive” pain or “nociceptor-mediated” pain occurs in response to the activation of a specific subset of peripheral sensory neurons (nociceptors) by intense or noxious stimuli. Nociceptive pain according to the invention includes mechanical pain (crushing, tearing, etc.) and chemical (iodine in a cut, chili powder in the eyes). Examples of nociceptive pains include but are not limited to traumatic or surgical pain, labor pain, sprains, hone fractures, burns, humps, bruises, injections, dental procedures, skin biopsies, and obstructions. Nociceptive pain includes visceral pain and somatic pain, in particular deep somatic pain and superficial somatic pain.

“Visceral pain” is diffuse, difficult to locate and often refers to a distant, usually superficial, structure. It may be accompanied by nausea and vomiting and may be described as sickening, deep, squeezing, and/or dull. Deep somatic pain is initiated by stimulation of nociceptors in ligaments, tendons, bones, blood vessels, fasciae and muscles, and is a dull, aching and poorly localized pain. Examples of deep somatic pain include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns.

The terms “injury-induced pain” encompass within the context of the present invention neuropathic pain, inflammatory pain and postoperative pain.

“Inflammatory pain” is a pain that occurs in the presence of tissue damage or inflammation including postoperative, post-traumatic pain, arthritic (rheumatoid or osteoarthritis) pain, pain associated with autoimmune disease (such as psoriasis) and pain associated with damage to joints, muscle and tendons as in axial low back pain. Inflammation is responsible for the sensitization of peripheral sensory neurons, leading to spontaneous pain and invalidating pain hypersensitivity. Acute or chronic pathological tissue inflammation strongly impacts on pain perception by sensitizing peripheral sensory neurons, giving rise to local and incapacitating pain hypersensitivity. Inflammatory mediators are known to enhance nociceptive primary afferent fibers excitability in part by modifying expression and/or function of ion channels present in nerve endings.

“Neuropathic pain” is a common type of chronic, non-malignant pain, which is the result of an injury or malfunction in the peripheral or central nervous system. Neuropathic pain may have different etiologies, and may occur, for example as a consequence of a trauma, surgery, herniation of an intervertebral disk, spinal cord injury, diabetes, infection with herpes zoster (shingles), HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs. A particular type of neuropathic pain is a «Chemotherapy-induced peripheral neuropathic pain» (CIPN) or a «Chemotherapy-induced neuropathic pain» (CINP). CINP or CIPN are among the most severe side effects of anticancer agents, such as platinum- and taxane-derived drugs (oxaliplatin, cisplatin, carboplatin and paclitaxel). CINP may even be a factor of interruption of treatment and consequently increase the risk of death. Neuropathic pain is often characterized by or responsible for the appearance of chronic allodynia (defined as pain resulting from a stimulus that does not ordinarily elicit a painful response, such as light touch) and/or hyperalgesia (defined as an increased sensitivity to a normally painful stimulus), and may persist for months or years beyond the apparent healing of any damaged tissues. Pain may also occur in patients with cancer, which may be due to multiple causes such as inflammation, compression, invasion, metastatic spread into bone or other tissues. Pain also includes migraine and a headache associated with the activation of sensory fibers innervating the meninges of the brain.

The products, in particular the peptides, set, cell or composition, of the invention may be used for preventing or treating a chronic pain, a neuropathic pain, a postoperative pain, an inflammatory pain, hyperalgesia, allodynia, or acute pain.

In another particular embodiment, the products, in particular the peptides, set, cell or composition, of the invention may be used for preventing or treating an arthritic pain, pain associated with damage to joints, pain related to Ehlers-Danlos syndrome, pain resulting from sun burn or pain resulting from ice burn

In particular, the products, in particular the peptides, set, cell or composition, of the invention may be used for preventing or treating, in particular treating, pain related to Ehlers-Danlos syndrome which comprises chronic muscle and/or bone and/or joint pain. The products, in particular the peptides, set, cell or composition, of the invention may also be used to prevent or treat, in particular treat, pain resulting from sun burn (i.e., that results from an overexposure to ultraviolet radiation) or pain resulting from ice burn (also called freezer burn or freeze-burn).

Preferably, the products, in particular the peptides, set, cell or composition, of the invention are used for preventing or treating injury-induced pain. More preferably, the products, in particular the peptides, set, cell or composition, of the invention are used for preventing or treating a neuropathic pain (such as a chemotherapy-induced neuropathic pain or a chemotherapy-induced peripheral neuropathic pain), a post-operative pain and/or an inflammatory pain.

Typically, the products, in particular the peptides, set, cell or composition, of the invention are used for preventing or treating chronic injury-induced pain. Typically, the products of the invention are used for preventing or treating chronic neuropathic pain such as chronic chemotherapy-induced peripheral neuropathic pain (CIPN), a chronic chemotherapy-induced neuropathic pain (CINP) or a chronic nerve injury-induced pain; chronic postoperative pain; and/or chronic inflammatory pain; chronic pain associated with or resulting from severe burns.

Within the context of the present invention, the term “treatment” or “treating” pain in a subject, designates delaying, stabilizing, curing, healing, alleviating, relieving, altering, ameliorating, improving, remedying or affecting any form of pain in a subject as described herein, or any disease or condition associated with pain, in particular acute, subacute or chronic pain (in particular any neuropathic condition associated with chronic pain resulting typically from a neuropathic pain, post-operative pain or inflammatory pain), or any symptom of such a disease or condition, after the exposition of the subject to, i.e., the application or administration of a suitable product or set of products of the invention, in particular i) a set of peptides (wherein the peptides are not linked to each other), preferably of the peptides of sequence SEQ ID NO: 1 and SEQ ID NO: 2 or of variants thereof as herein defined, ii) a nucleic acid or set of nucleic acids allowing the expression of such a set of peptides which am not linked to each other, iii) an expression cassette or a set of expression cassettes comprising a set of nucleic acids as herein defined, iv) a vector or a set of vectors allowing the expression of such a set of peptides which are not linked to each other, v) a cell or set of cells allowing the expression of such a set of peptides which are not linked to each other, or vi) a composition according to the invention comprising such peptides, nucleic acids, expression cassette(s), vector(s) and/or cell(s).

The term “treatment” or “treating” also refers to any indicator of success in the treatment of pain (which may be associated with any injury, pathology or condition), including any objective or subjective parameter such as abatement, remission, slowing progression or severity, stabilization, diminishing of symptoms of pain, or making it tolerable or more tolerable to the subject. The term “treating” pain, also includes increasing pain tolerance and/or decreasing perceived pain. In particular aspects, the methods, compounds and compositions of the invention are for increasing pain tolerance and/or for decreasing perceived pain. As used herein, the terms “pain tolerance” refers to the amount of pain that a subject can perceive and withstand before breaking down emotionally and/or physically. Pain tolerance is distinct from pain threshold (the minimum mechanical stimulus necessary to produce pain). As used herein, “increasing pain tolerance” generally refers to a situation where a subject can develop a greater pain tolerance (that is, less perceived pain) when compared to a previous state, for instance, following administration of suitable peptides of sequence SEQ ID NO: 1 and of sequence SEQ ID NO:2 or of variants thereof or of a composition comprising said peptides or variants to a subject.

Within the context of this invention, “preventing” or “prevention” in relation to pain in a subject, refers to at least the reduction of likelihood of the risk of acquiring (or susceptibility to acquire) any kind of pain by a subject, after the exposition of the subject to, i.e., the application or administration of product(s) of the invention, in particular i) a suitable set of peptides (wherein the peptides are not linked to each other), preferably of the peptides of sequence SEQ ID NO: 1 and SEQ ID NO: 2 or of variants (typically functional variants) thereof as herein defined, ii) a nucleic acid or set of nucleic acids allowing the expression of such a set of peptides which are not linked to each other, iii) an expression cassette or a set of expression cassettes comprising a set of nucleic acids as herein defined, iv) a vector or a set of vectors allowing the expression of such a set of peptides which are not linked to each other, v) a cell or set of cells allowing the expression of such a set of peptides which are not linked to each other, or of vi) a composition according to the invention comprising such peptides, nucleic acids, expression cassette(s), vector(s) and/or cell(s). For example, “preventing” includes causing at least one of the clinical symptoms of pain not to develop in a subject that may be exposed to or predisposed to, but does not yet experience or display symptoms of pain.

In the context of the invention, a “subject” or “patient” designates an animal, in particular a mammal in need of treatment for a disease or disorder or a symptom thereof. The subject may be a subject having been diagnosed as suffering of a disease or disorder, or determined to be at risk of developing a disease or disorder, said disease or disorder being known to cause the subject to feel pain. In particular example, the subject is diagnosed or suffering from pain, such as acute pain and/or sub-acute pain or chronic pain, including neuropathic pain, postoperative pain, inflammatory pain, hyperalgesia and/or allodynia.

In a particular aspect, the subject is a human-being.

In another particular aspect, the subject is an animal, in particular a domestic or breeding animal, in particular a horse, a dog, a cat, a cow, etc.

In another particular aspect, the subject has at least one mutated allele in the myo1A gene.

### Peptides

The inventors herein identify a novel set of components, in particular a set of peptides (wherein said peptides are not linked to each other) that can prevent or treat pain when used in combination, either simultaneously or sequentially, preferably simultaneously, in particular that can reverse mechanical hypersensitivity in the context of injured or inflamed nervous system. Without being bound by any theory, inventors believe that by modulating excitability of spinal cord network, these peptides exhibit either antalgic or analgesic activity, in particular antalgic activity, by specifically targeting mechanically and/or chemically induced nociceptive signals.

The herein described peptides are, i.e., consist of, specific fragments from the human TAFA-4 mature protein (SEQ ID NO: 5:

SQHLRGHAGHHQIKQGTCEVVAVHRCCNKNRIEERSQTVKCSCFPGQVAGTTRAQPSCVE ASIVIQKWWCHMNPCLEGEDCKVLPDYSGWSCSSGNKVKTTKVTR) of 105 amino acid residues (resulting from the cleavage of the signal sequence and identified in the public data base under the accession #NP_0011005527 or under the Genbank accession AAP92409 as disclosed by Tang et al., 2004).

Inventors in particular herein describe for the first time a set of products/components, in particular a set of isolated or synthetic peptides, comprising, consisting of, or consisting essentially of, i) a first peptide of SEQ ID NO: 1 (CFPGQVAGTTRAQPSCVEASIVIQKWW, i.e., Cys-Phe-Pro-Gly-Gln-Val-Ala-Gly-Thr-Thr-Arg-Ala-Gln-Pro-Ser-Cys-Val-Glu-Ala-Ser-Ile-Val-Ile-Gln-Lys-Trp-Trp) or a peptide having at least 90% identity to SEQ ID NO: 1 (also herein identified as “variant”, “peptide variant” or “variant of the peptide” of sequence SEQ ID NO: 1), and ii) a second peptide of SEQ ID NO: 2 (CHMNPCLEGEDCKVLPDYSGWSCSSGNKVKTTKVTR, i.e., Cys-His-Met-Asn-Pro-Cys-Leu-Glu-Gly-Glu-Asp-Cys-Lys-Val-Leu-Pro-Asp-Tyr-Ser-Gly-Tip-Ser-Cys-Ser-Ser-Gly-Asn-Lys-Val-Lys-Thr-Thr-Lys-Val-Thr-Arg) or a peptide having at least 90% identity to SEQ ID NO: 2 (also herein identified as “variant”, “peptide variant” or “variant of the peptide” of sequence SEQ ID NO: 2), wherein said (at least two) peptides are not linked to each other.

Peptides having at least 90% identity to a particular amino acid/peptide sequence are herein identified as “variant”, “peptide variant” or “variant of the peptide” having said sequence. In a preferred aspect, any variant present in the set, or in a composition comprising/expressing such a set, is a functional variant as further explained herein below.

The terms “consisting essentially of” have the meaning generally ascribed to them e.g., they allow for elements not explicitly recited, but exclude elements that are found in the prior art or that affect a basic or novel characteristic of the invention. As such, a set of peptides “consisting essentially of” specified sequences refers to peptides comprising or consisting of these sequences (the sequences being not linked to each other) and that include other features not essential to the activity of the peptide.

The peptide of SEQ ID NO: 1 consists of the 27 C-terminal amino acid residues of the human TAFA-4 mature protein, and may be identified as amino acids 78-104 of UniProt accession #Q96LR4.

The peptide of SEQ ID NO: 2 consists of 36 amino acid residues of the human TAFA-4 mature protein, and may be identified as amino acids 105-140 of UniProt accession #Q96LR4.

In a particular aspect, the peptide of SEQ ID NO: 1 and/or the peptide of SEQ ID NO: 2 is/are acetylated in the N-terminal position and/amidated in the C-terminal position.

The two amino acid residues Q (Glu) appearing in bold at position 13 within SEQ ID NO: 1: CFPGQVAGTTRAQPSCVEASIVIQKWW, and Y (Tyr) appearing in bold at position 18 within SEQ ID NO: 2: CHMNPCLEGEDCKVLPDYSGWSCSSGNKVKTTKVTR (with reference to positions set forth respectively in SEQ ID NO:1 and SEQ ID NO: 2), are believed by inventors to be implied in the biological activity of the peptides.

Variants of peptides of SEQ ID NO: 1 and SEQ ID NO: 2 are also herein disclosed for the first time by inventors. Variants are intended to designate a peptide derived from the peptide of SEQ ID NO: 1 or from the peptide of SEQ ID NO: 2 and resulting from the deletion or addition of one or more amino acids and/or substitution of one or more amino acids at one or more sites in said peptides of SEQ ID NO:1 or SEQ ID NO: 2. A variant will be considered a “functional variant” if the variant substantially retains the functional activity of the peptide of SEQ ID NO: 1 or of SEQ ID NO: 2, or even improves said functional activity allowing the prevention or treatment pain. The activity may be measured using functional assays such behavioral assays as performed in the experimental part.

A variant of a peptide usable in the context of the present invention may have at least 90% as explained herein above, for example (at least) 90.5%, 91%, 92%, 92.1%, 93%, 93.7%, 94%, 95%, 95.2%, 96%, 96.8%, 97%, 98%, 98.4% or 99% sequence identity to the sequence encoding said peptide, for example to SEQ ID NO: 1 or SEQ ID NO: 2.

In a particular aspect, the variant having at least 90% identity to SEQ ID NO: 1 or SEQ ID NO: 2 modulates excitability of spinal cord interneurons (preferably spinal cord lamina IIi interneurons). In a preferred aspect, the variant is a peptide comprising one or more point mutation(s) (e.g. 2, 3, 4, 5 or 6 point mutations) that add, delete or substitute any of the amino acids present in the peptide sequence of interest as herein described, such as in particular SEQ ID NO: 1 or SEQ ID NO: 2.

Regarding specifically the peptide variants having at least 90% identity to SEQ ID NO: 1 or SEQ ID NO: 2, the preceding paragraph applies with the proviso that amino acid residue Q at position 13 in SEQ ID NO: 1 remains unchanged in the amino acid sequence of the peptide having at least 90% identity to SEQ ID NO: 1, and with the proviso that amino acid residue Y at position 18 in SEQ ID NO: 2 either remains unchanged in the amino acid sequence of the peptide having at least 90% identity to SEQ ID NO: 2 or is replaced by a serine (S) (positions 13 and 18 being with reference to positions set forth respectively in SEQ ID NO: 1 and SEQ ID NO: 2). A peptide having an amino acid residue S at position 18 is of SEQ ID NO: 6 (CHMNPCLEGEDCKVLPDSSGWSCSSGNKVKTTKVTR, i.e., Cys-His-Met-Asn-Pro-Cys-Leu-Glu-Gly-Glu-Asp-Cys-Lys-Val-Leu-Pro-Asp-Ser-Ser-Gly-Trp-Ser-Cys-Ser-Ser-Gly-Asn-Lys-Val-Lys-Thr-Thr-Lys-Val-Thr-Arg).

Thus, in a preferred aspect of the invention, the amino acid Q (glutamine residue) at position 13 in the peptide of SEQ ID NO: 1 (/with reference to position set forth in SEQ ID NO:1) remains unchanged in the amino acid sequence of the variant of the peptide having at least 90% identity to SEQ ID NO: 1. Similarly, in another preferred aspect, the amino acid Y (tyrosine residue) at position 18 in the peptide of SEQ ID NO: 2 (/with reference to position set forth in SEQ ID NO:2) either remains unchanged in the amino acid sequence of the variant of the peptide having at least 90% identity to SEQ ID NO: 2 or is replaced by a S (serine).

In another preferred aspect of the invention, the amino acid Q at position 13 (with reference to SEQ ID NO:1) remains unchanged in the amino acid sequence of the variant of the peptide having at least 90% identity to SEQ ID NO: 1 and the amino acid Y at position 18 (with reference to SEQ ID NO:2) either remains unchanged in the amino acid sequence of the variant of the peptide having at least 90% identity to SEQ ID NO: 2 or is replaced by a serine (S).

In another particular aspect, a variant of a peptide of SEQ ID NO: 1 usable in the context of the present invention may have at least 88.8%, 92.5%, 96.2% sequence identity to the peptide sequence of SEQ ID NO: 1, preferably with the proviso described herein above concerning amino acid residue Q at position 13 with reference to SEQ ID NO: 1. In another particular aspect, a variant of a peptide of SEQ ID NO: 2 usable in the context of the present invention may have at least 91.6%, 94.4%, 97.2% sequence identity to the peptide sequence of SEQ ID NO: 2, preferably with the proviso described herein above concerning amino acid residue Y at position 18 with reference to SEQ ID NO: 2. Preferably, said variants modulates excitability of spinal cord interneurons (preferably spinal cord lamina IIi interneurons).

In a particular aspect, the variant is a peptide comprising one or more point mutations (e.g. 1, 2, or 3point mutations) that add, delete or substitute any of the amino acids present in the peptide sequence of interest as herein described, such as in particular SEQ ID NO: 1 or SEQ ID NO: 2, with the proviso described herein above concerning amino acid residues Q at position 13 with reference to SEQ ID NO: 1, and Y at position 18 with reference to SEQ ID NO: 2.

In a particular aspect, the one or more deletions are at the N terminus of the sequence of interest such as SEQ ID NO: 1 or SEQ ID NO: 2. In another aspect, the one or more deletions are at the C terminus, or in any other position of the sequence of interest such as SEQ ID NO: 1 or SEQ ID NO: 2, with the proviso described herein above in relation with SEQ ID NO: 1 and SEQ ID NO: 2 concerning amino acid residues Q at position 13 with reference to SEQ ID NO: 1, and Y at position 18 with reference to SEQ ID NO: 2. In again another aspect, the two or more deletions are at both the N and C terminus of the sequence of interest such as SEQ ID NO: 1 or SEQ ID NO: 2. Such deletions at the N and/or C terminus for example of the peptide of SEQ ID NO: 1 or SEQ ID NO: 2, or in the core thereof, may result in truncated human peptide variants of SEQ ID NO: 1 or SEQ ID NO: 2.

In addition, any peptide of the set of peptides according to the present invention may be fused to another peptide or protein to form a conjugate usable in the methods herein described, so long as said “another peptide or protein” is not one of the “at least two” peptides of the set of the invention. Indeed, the peptides of the herein described set of peptides (active ingredients of the set) should not be linked to each other. The term “conjugate” in this context refers to an engineered fusion construct combining the biological functions of two molecules within a single polypeptide, in particular a peptide of SEQ ID NO: 1 or 2 or a variant thereof, for example capable of modulating excitability of a nociceptor or an interneuron, together with for example a polypeptide which specifically interacts or binds to a target cell.

The present invention also relates to a nucleic acid encoding a peptide of the set of the invention, for example a peptide of sequence SEQ ID NO: 1, a peptide of sequence SEQ ID NO:2, or a variant thereof as herein described. For example, any sequence coding for the peptide of SEQ ID NO: 1, for the peptide of SEQ ID NO:2, or for a variant thereof, are encompassed by the present invention as well as similar sequences resulting from the degeneration of the genetic code. In a particular aspect, the nucleic acid encoding a peptide as herein described comprises, consists of, or consists essentially of, a sequence of SEQ ID NO: 3: TGCTTCCCGGGACAGGTGGCGGGCACAACTCGGGCTCAACCTTCTTGTGTTGAAGCTT CCATTGTGATTCAGAAATGGTGG, or SEQ ID NO: 4: TGTCACATGAATCCGTGTTTGGAAGGAGAGGATGTAAAGTGCTGCCAGATTACTCAG GTTGGTCCTGTAGCAGTGGCAATAAAGTCAAAACTACGAAGGTAACGCGG.

Inventors in particular herein describe a set of nucleic acids allowing the expression of (at least two) peptides which are not linked to each other, wherein a (first) nucleic acid comprises a sequence encoding a (first) peptide of interest, for example a peptide of SEQ ID NO: 1 or a peptide having at least 90% identity to SEQ ID NO: 1, and a (second) nucleic acid comprises a sequence encoding a (second) peptide of interest, for example a peptide of SEQ ID NO: 2 or a peptide having at least 90% identity to SEQ ID NO: 2. Inventors also herein describe a composition comprising such a set of nucleic acids, preferably together with an acceptable support, carrier, excipient or vehicle as herein described.

The above-mentioned nucleic acid sequences encoding the peptides of SEQ ID NO: 1, SEQ ID NO: 2, or a variant thereof, can be flanked by regulatory sequences for controlling its expression in an appropriate host cell.

In another aspect, the present invention also relates to an expression cassette comprising, in this order from 5′ to 3′:
a promoter,
a nucleic acid sequence encoding a peptide of interest such as SEQ ID NO: 1, SEQ ID NO: 2, or a variant thereof as herein described; and
termination signals, such as for example a polyadenylation signal.

The above-mentioned nucleic acid sequences encoding a peptide of interest, or the expression cassette disclosed herein can be flanked by sequences suitable for their packaging into a vector that optimizes its transcription and/or translation in a cell.

In another aspect, the present invention also relates to a vector permitting expression of a peptide or a set of peptides as herein described. The present invention also relates to a vector comprising a set of nucleic acids allowing the expression of (at least two) peptides which are not linked to each other, or a set of expression cassettes as herein defined.

In a further aspect, the present invention relates to a set of vectors allowing the expression of (at least two peptides) which are not linked to each other, wherein a (first) vector comprises a sequence encoding a (first) peptide of interest, for example of SEQ ID NO: 1 or a peptide having at least 90% identity to SEQ ID NO: 1, and a (second) vector comprises a sequence encoding another (a second) peptide of interest, for example a peptide of SEQ ID NO: 2 or a peptide having at least 90% identity to SEQ ID NO: 2. Inventors also herein describe a composition comprising such a set of vectors, preferably together with an acceptable support, carrier, excipient or vehicle as herein described.

### Methods for Producing the Peptide

To allow expression in host cells, the nucleic acid encoding (a) peptide(s) (or variant(s) thereof) as herein described can be present in (a) vector(s) and, after introduction of said vector(s) into a suitable host cell, the sequence(s) can be expressed to produce the encoded peptide(s) herein described according to standard cloning and expression techniques, which are well-known in the art (e.g., as described in Sambrook, J., Fritsh, E. F., and Maniatis, T. Molecular Cloning: A Laboratory Manual 2nd, ed., Cold Spring Harbor Laboratory. Cold Spring Harbor Laboratory Press, Cold Spring Harbor, N.Y., 1989). Various expression vectors can be employed to express a polynucleotide sequence encoding a peptide as herein described. An expression vector that can be used in the present invention includes non-exhaustively an eukaryotic expression vector, in particular a mammalian expression vector, a virus-based expression vector, a baculovirus expression vector, a plant expression vector and any plasmid expression vector in order to produce anyone of the herein described peptide in a host cell. The expression vector could also be a vector allowing the expression of the peptide(s) in a bacterial system. When a single nucleic acid encodes, or a single vector allows the expression, of several peptides, the resulting peptides are not linked to each other.

The choice of the expression vector depends on the intended host cells in which the vector is to be expressed. This choice will be easily made by the skilled person. The present invention also relates to a cell, in particular a host cell, comprising a nucleic acid sequence encoding a peptide as herein described. A (host) cell modified using a vector as herein described is also herein disclosed for the first time by inventors.

Herein disclosed in particular is a cell allowing the expression of (at least two) peptides (or variants thereof) as herein described which are not linked to each other. This cell typically comprises a (first) nucleic acid sequence or vector encoding a peptide of interest, such as a peptide of SEQ ID NO: 1 or a peptide having at least 90% identity to SEQ ID NO: 1, and a (second) nucleic acid sequence or vector encoding a (second/different) peptide of interest, such as a peptide of SEQ ID NO: 2 or a peptide having at least 90% identity to SEQ ID NO: 2. Also disclosed is a composition comprising such a cell, preferably together with an acceptable support, carrier, excipient or vehicle as herein described.

Herein disclosed in particular is a set of cells allowing the expression of (at least two) peptides (or variants thereof) as herein described which are not linked to each other. Each cell typically comprises a nucleic acid sequence or vector encoding a peptide of interest, such as a peptide of SEQ TD NO: 1 or SEQ ID NO: 2, or a peptide having at least 90% identity to SEQ ID NO: 1 or SEQ ID NO: 2. Also disclosed is a composition comprising such cells, preferably with an acceptable support, carrier, excipient or vehicle as herein described.

It is possible to express the peptide(s) of the present invention, or variants thereof, in cither prokaryotic or eukaryotic host cells. Representative host cells include many E. coli strains, mammalian cell lines, such as for example CHO, CHO-K1, and HEK293; insect cells, such as Sf9 cells; and yeast cells, such as S. cerevisiae and P. pastoris.

The nucleic acid or vector herein described may be transfected into a host cell by standard techniques commonly used for the introduction of exogenous DNA into a prokaryotic or eukaryotic host cell, such as for example electroporation, calcium phosphate precipitation, DEAE-dextran transfection, etc. Alternatively, the nucleic acid or vector herein described may be delivered into a host cell by transduction using a viral-based vector.

Purity may be measured by any appropriate standard method, for example, by column chromatography, thin layer chromatography, or high-performance liquid chromatography (HPLC) analysis. “Isolated” or “synthetic” peptide also defines a degree of sterility that is safe for administration to a human subject, e.g., lacking infectious or toxic agents.

The peptides used in the compositions and methods herein described can also be produced by solid phase synthesis technique. Direct chemical synthesis of peptides can be accomplished by methods very well-known by the person skilled in the art, such as Native Chemical Ligation (NCL). This chemical approach consists of coupling of unprotected peptide fragments: peptide with N-terminal cysteine reacts with a C-terminal thioester peptide. This transthioesterification is rapidly followed by an intramolecular S,N-acyl shift that leads to the formation of a native amide bond at the ligation site.

### Gene Therapy

By cloning the nucleic acid sequences encoding the peptides herein described into appropriate vector(s), the inventors also herein provide novel gene delivery tools, in particular genetic constructs (such as for example a cassette of expression or a vector) for gene therapy of pain, in particular acute, subacute or chronic pain, preferably chronic pain. Gene therapy may be employed to allow the endogenous production of TAFA-4-peptides or variants thereof by specific cells in a subject. Gene therapy can either occur in vivo or ex vivo. Ex vivo gene therapy requires the isolation and purification of at least a sample of subject's cells, the introduction of at least one nucleic acid sequence (i.e. transgene) encoding a set of peptides as herein described into the isolated cells, and the introduction of the genetically altered/modified cells back into the subject. In contrast, in in vivo gene therapy, the transgene is typically packaged for administration to the subject. Gene delivery constructs may be either non-viral or viral. Preferably, the genetic construct herein described is prepared with viral elements, viral vectors and/or any viral packaging system(s) that may be used to express a transgene/coding sequence (i.e. a nucleic acid sequence encoding the peptides of the set of interest or variants thereof) in a targeted tissue/cell. The viral vectors can incorporate any suitable promoter and other transcription regulator that allow or facilitate expression of the transgene product in the targeted tissue/cell. The viral packaging system is preferably adapted to the targeted cell. Once into the target cell, such a system facilitates the delivery to the targeted tissue. The viral vector(s) usable in the methods herein described is(are) preferably (a) replication-deficient virus(es), such as for example (an) adenovirus or adeno-associated virus (AAV) vector(s).

In some aspects, the present invention relates to a recombinant adeno-associated virus (AAV) comprising in its genome, a nucleic acid sequence encoding at least one peptide of the set of interest, preferably at least two peptides, for example a peptide of SEQ ID NO: land/or a peptide of SEQ ID NO: 2, or variant(s) thereof. The coding part of the nucleic acid is typically operably linked to a promoter.

To date, at least dozens different serotypes of AAVs with variations in their surface properties have been isolated from human or non-human primates (NHP) and characterized. The term “serotype” allows the skilled person to distinguish between AAV having serologically different capsids. Serologic distinctiveness is determined on the basis of the lack of cross-reactivity between antibodies to one AAV serotype as compared to other AAV serotypes. The rAAV herein described, also named rAAV vector or rAAV particle, may have any one of the following known serotypes, i.e. may be selected for example from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. The rAAV vector may have enhanced tropism for a particular cell, tissue or organ. In the context of an administration by oral route, the rAAV vector may have enhanced tropism for stomach, small intestine or colon tissue and more specifically for cells that constitute these tissues, in particular for epithelial cells, such as for example enterocytes, Goblet cells, enteroendrocine cells, Paneth cells, or Tuft cells. For targeted cells located in, or targeting cells delivered to, the gut, anyone of AAV 4, AAV7, AAV8, AAV9 or AAV10 may be selected as particularly efficient.

Gene therapy vectors or cassettes of the present invention may be produced by methods well-known by the skilled person and previously described, e.g., in PCT Patent Application Publication No. WO03042397 and U.S. Pat. No. 6,632,670.

The present description also relates to a method for producing a recombinant vector, for example a viral vector such as an AAV, comprising:
a) culturing a cell that has been transfected/transduced with a recombinant vector described herein; and
b) recovering the recombinant vector from the supernatant of the transfected/transduced cells.

Gene therapy vector of the present invention may be produced by the transfection of two or three plasmids into a 293 or 293T human embryonic kidney cell line. In a particular aspect, the DNA(s) coding for the therapeutic peptides is (are) provided by one plasmid; the capsid proteins and replication genes issued from AAV of one or more serotypes, and helper functions issued for example from an adenovirus are all provided in trans by a second plasmid. In another particular aspect, the DNA coding for the peptides of SEQ ID NO: 1 and SEQ ID NO: 2 or variants thereof is provided by one plasmid; the capsid proteins and replication genes issued from AAV of one or more serotypes are provided in trans by a second plasmid, and helper functions issued for example from adenovirus are provided by a third plasmid. In a particular aspect, the first plasmid comprises an expression cassette comprising a nucleic acid sequence encoding a peptide of SEQ ID NO: 1 and a peptide of SEQ ID NO: 2 or variants thereof as herein described operably linked to a promoter, including two flanking inverted terminal repeats (ITRs). In another particular aspect, the DNA coding for each of the therapeutic peptides are provided via two distinct plasmids, a first plasmid comprising a nucleic acid sequence encoding a peptide of SEQ ID NO: 1 or a variant thereof, and a second plasmid comprising a nucleic acid sequence encoding a peptide of SEQ ID NO: 2 or variants thereof.

Following cell culture, the gene therapy vector(s) may be for example released from cells by freeze thaw cycles, and purified by any method well known by the skilled person, such as for example by using an iodixanol step gradient followed by ion exchange chromatography on Hi-Trap QHP columns. Then, the resulting gene therapy vector(s) may be concentrated by spin column, and the purified vector(s) may be stored frozen (at or below −60° C.), e.g., in phosphate buffered saline.

Related aspects of the invention include a host cell transfected or transduced with at least one recombinant vector, for example transduced with at least one AAV vector, described herein; or a set of cells transfected with a vector or a set of vectors comprising DNA coding for the therapeutic peptides or variants thereof as defined herein. Further related aspects of the invention include any nucleic acid molecule comprising, or consisting (essentially of), the genome of a recombinant vector, for example AAV, described herein.

### Compositions

The herein described composition comprising a set of (at least two) peptides not linked to each other as herein described, or a product as herein described (for example a nucleic acid, a cell, a set of nucleic acids or a set of cells) allowing the expression of said (at least two) peptides, may be a pharmaceutical, therapeutic, prophylactic, or veterinary composition. The composition may also be (possibly simultaneously) a dietetically/dietarily composition.

A particular composition herein described comprises a peptide of sequence SEQ ID NO: 1 or a (typically functional) variant thereof (i.e., a peptide having at least 90% identity to SEQ ID NO: 1), and a peptide of sequence SEQ ID NO: 2 or a (typically functional) variant thereof (i.e., a peptide having at least 90% identity to SEQ ID NO: 2), wherein said (at least two) peptides are not linked to each other.

The set of two or more peptides herein described are advantageously formulated in order for the peptides to be used in combination, either separately or simultaneously. Thus, each peptide of the set can be prepared in a separate form or in the form of a composition comprising a mix of at least two peptides of the set. The composition may be a dietary/dietetical composition and/or a pharmaceutical composition, and may be used in a therapeutic or prophylactic method as herein described.

In a particular aspect, the present invention relates to a composition comprising at least two peptides or variants thereof which are not linked to each other, typically a set of (at least two) peptides which are not linked to each other, a nucleic acid or a set of nucleic acids encoding (at least two) peptides which are not linked to each other, an expression cassette or a set of expression cassettes allowing the expression of (at least two) peptides which are not linked to each other, a vector or set of vectors allowing the expression of (at least two) peptides which are not linked to each other, or a cell or set of cells allowing the expression of (at least two) peptides which are not linked to each other, as herein described, and preferably an acceptable support, carrier, excipient or vehicle as herein described, in particular a dietarily and/or pharmaceutically acceptable support, carrier, excipient or vehicle.

The terms “dietarily-acceptable support” relate to a carrier permitting the subject to ingest and digest without risk the composition comprising a peptide or set of peptides, a nucleic acid sequence or set of nucleic acid sequences, an expression cassette or set of expression cassettes, a vector or set of vectors, or a cell or set of cells as herein described, and capable of protecting said peptide(s) from any attack, in particular related to food digestion, that could alter it (them) before it (they) produce(s) its (their) therapeutic action(s) on the correct site and at the correct moment depending on the nature and localization of pain.

In a particular aspect where the composition comprises two peptides, the first and the second peptides can be present in the composition of the invention in equal or different proportions. In a particular composition, the first and second peptides are present in the composition in equal (equimolar) proportions, i.e. each peptide represents 50% of the peptide component of the composition, meaning that the composition contains the same number of moles of each of the two peptides.

Another particular peptide composition, may comprise a greater proportion of a particular peptide, or of particular peptides relative to another peptide or to other peptides. For example, a particular peptide may represent at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% of the peptide component of the composition.

In the context of oral administration, the composition may further comprise at least one gastrointestinal protective agent, preferably an acid inhibitor, present in an amount effective to raise the gastric pH of a subject to at least 2, to at least 3, to at least 4, and more preferably to at least 5 or 6. The term “acid inhibitor” refers to agents that inhibit gastric acid secretion and increase gastric pH. The acid inhibitor may include, but is not limited to H₂ blockers including cimetidine, ranitidine, ebrotidine, pabutidine, lafutidine, loxtidine, famotidine; proton pump inhibitors including omeprazole, esomeprazole, pantoprazole, lansoprazole, dexlansoprazole, rabeprazole, pariprazole, leminoprazole and tenatoprazole; or any combination thereof.

The “pharmaceutically-acceptable support/vehicle/carrier” can be a diluent, adjuvant, or excipient with which the active agent(s) (i.e. the at least two peptides or variants thereof of the invention and optionally any additional distinct active agent(s)) is(are) administered. Such a pharmaceutical carrier can be a sterile liquid, such as water or oil, including those of petroleum, animal, vegetable or of synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The pharmaceutically-acceptable support/vehicle/carrier may consist in or comprise ascorbic acid and/or an ascorbic acid salt. Examples of ascorbic acid salts are Magnesium Ascorbate, Sodium Ascorbate, Calcium Ascorbate and Ascorbyl Palmitate.

In one embodiment, the composition of the present invention comprises ascorbic acid or a ascorbic acid salt.

When the composition, for example the pharmaceutical composition, is adapted for oral administration, the tablets or capsules can be prepared by conventional means with pharmaceutically acceptable excipients such as binding agents (e.g. pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by any method well known in the art. Liquid preparations for oral administration may take the form of, for example, solutions, syrups or suspensions, or they may be presented as a dry product for reconstitution with water or another suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The composition of the present invention, for example the pharmaceutic, prophylactic or therapeutic composition, may further comprise at least one additional (distinct) active compound. Preferably, the additional active compound is an active agent efficient against pain. By “efficient against pain” is meant an active agent having analgesic or antalgic properties (measurably felt by the subject). More preferably, the additional active compound is a steroidal anti-inflammatory drug (SAID), a nonsteroidal anti-inflammatory drug (NSAID) or an opioid drug.

The SAID may include, but is not limited to, hydrocortisone, cortisone, ethamethasoneb, prednisone, prednisolone, triamcinolone, dexamethasone, fludrocortisone, or any combination thereof.

The NSAID may include, but is not limited to, celecoxib, rofecoxib, lumiracoxib, valdecoxib, parecoxib, etoricoxib, CS-502, JTE-522, L-745,337, NS398, aspirin, acetaminophen (considered to be an NSAID for the purposes of the present disclosure), ibuprofen, flurbiprofen, ketoprofen, naproxen, oxaprozin, etodolac, indomethacin, ketorolac, lornoxicam, meloxicam, piroxicam, droxicam, tenoxicam, nabumetone, diclofenac, meclofenamate, mefenamic acid, diflunisal, sulindac, tolmetin, fenoprofen, suprofen, benoxaprofen, aceclofenac, tolfenamic acid, oxyphenbutazone, azapropazone, phenylbutazone, or any combination thereof.

The opioid drug may include, but is not limited to, (dextro)propoxyphene, A-methylfentanyl, alfentanil, allylprodine, bezitramide, buprenorphine, butorphanol, carfentanyl, desmethylprodine, dextromoramide, dezocine, diacetylmorphine, dihydrocodeinone, dihydroetorphine, dimorphone, diphenoxylate, dipipanone, etorphine, fentanyl, ketobemidone, lefetamine, levacetylmethadol, levomethorphan, levorphanol, loperamide, meperidine, meptazinol, methadone, methylmorphine, morphine, nalbuphine, nicomorphine, ohmefentanyl, oripavine, oxycodone, oxymorphone, PEPAP, paramorphine, pentazocine, phenazocine, piritramide, prodine, remifentanil, sufentanil, tapentadol, tilidine, tramadol, or opioid antagonists such as nalmefene, naloxone, naltrexone or any combination thereof.

### Therapeutic and Prophylactic Uses

The present description discloses peptides, nucleic acid(s), expression cassette(s), vector(s), cell(s), and sets thereof, or any combination thereof, as well as a composition comprising such peptides, nucleic acid(s), expression cassette(s), vector(s), cell(s), and/or set(s) thereof, as herein above described, for use as active prophylactic or therapeutic ingredient(s), preferably for use as a pharmaceutical composition, drug or medicament.

In a particular aspect, the peptides, in particular the isolated or synthetic peptide of SEQ ID NO:1 and/or the isolated or synthetic peptide of SEQ ID NO:2, preferably the set of said peptides, any set allowing the expression of such peptides, or any composition comprising such peptides, nucleic acid(s), expression cassette(s), vector(s), cell(s), and/or set(s) as herein described, is/are for use as a pharmaceutical composition, drug or medicament, in particular for use for preventing or treating pain.

In another aspect, the present description relates to peptide(s), nucleic acid(s), expression cassette(s), vector(s) or cell(s), as herein described, and/or set(s) as herein described, for use as active ingredient(s)/agent(s) for preventing or treating pain in a subject in need thereof.

The present description relates in particular to a herein disclosed product or set of any of the herein disclosed products, for example a set of cell as herein described, typically to a set of peptides or cells comprising or expressing such a set of peptides, for use [as (an) active(s) ingredient(s)] for preventing or treating pain in a subject in need thereof.

In a particular aspect, inventors herein describe the use of any product of the invention as herein described such as peptide(s), nucleic acid(s), expression cassette(s), vector(s), cell(s), as herein described, and corresponding sets thereof, or any combination thereof as herein described, for the manufacture of a pharmaceutical composition, drug or medicament for the prevention or treatment of pain in a subject in need thereof. They also herein describe corresponding methods for preventing or treating pain, said methods comprising typically a step of administering said peptide(s), nucleic acid(s), expression cassette(s), vector(s), cell(s), set(s) and/or composition(s) as herein described to a subject in need thereof.

The present description in particular provides a method of preventing or treating pain in a subject in need thereof comprising a step of administering a (therapeutic) effective amount of a product such as in particular a set of peptides and/or composition as herein described, preferably a set of peptides comprising the peptide of SEQ ID NO: 1 or a variant thereof having at least 90% identity to SEQ ID NO: 1, and the peptide of SEQ ID NO: 2 or a variant thereof having at least 90% identity to SEQ ID NO: 2, or a composition comprising such a set.

In a particular aspect, inventors herein describe the use of any product of the invention as herein described such as peptide(s), nucleic acid(s), expression cassette(s), vector(s), cell(s), as herein described, and corresponding sets thereof, or any combination thereof or composition as herein described, for preventing or treating pain, in particular acute, sub-acute or chronic pain, preferably chronic pain.

Preferably, the pain is a neuropathic pain (such as chemotherapy-induced peripheral neuropathic pain or a chemotherapy-induced neuropathic pain), a postoperative pain, an inflammatory pain, hyperalgesia, or allodynia.

In a particular aspect, inventors herein describe the use of any product of the invention as herein described such as peptide(s), nucleic acid(s), expression cassette(s), vector(s), cell(s), as herein described, and/or a set(s) as herein described thereof, or any combination thereof or composition as herein described, for preventing or treating neuropathic pain (such as chemotherapy-induced peripheral neuropathic pain or a chemotherapy-induced neuropathic pain), post-operative pain or inflammatory pain.

In a particular aspect, inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating chronic neuropathic pain (such as a chronic chemotherapy-induced peripheral neuropathic pain or a chronic chemotherapy-induced neuropathic pain), chronic post-operative pain or chronic inflammatory pain.

In a more particular aspect, the inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating an osteoarthritis pain.

In a particular aspect, inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating hyperalgesia, in particular thermal (such as heat or cold hyperalgesia, preferably cold hyperalgesia) or mechanical hyperalgesia, preferably mechanical hyperalgesia even more preferably injury-induced mechanical hyperalgesia.

In another particular aspect, inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating allodynia, in particular mechanical allodynia. Preferably, the mechanical allodynia is a nerve injury-induced mechanical allodynia or a mechanical allodynia of the static type.

In a further particular aspect, inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating allodynia, in particular thermal allodynia, such as heat allodynia (i.e. perceived pain to a normally non-painful warm stimulus) or cold allodynia (i.e. perceived pain to a normally non-painful cold stimulus), preferably cold allodynia.

In a particular aspect, inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating mechanical hypersensitivity (also referred as hypersensitivity to mechanical stimuli or simply as hyperalgesia), preferably injury-induced mechanical hypersensitivity, in a subject in need thereof.

In a particular aspect, the inventors herein describe the use of any product of the invention as herein described such as peptide(s), nucleic acid(s), expression cassette(s), vector(s), cell(s), as herein described, and corresponding sets thereof, or any composition thereof as herein described, for preventing or treating pain resulting from sun burn or pain resulting from ice burn.

Treatment may result in improvements in the sensations of one or more of touch, burning or coldness, “pins and needles”, numbness, itching, excruciating pain and difficulty to correctly sense temperature. In a particular aspect, the treatment eliminates pain. In another particular aspect, the treatment reduces the symptoms of pain, in particular those of neuropathic pain (allodynia and/or hyperalgesia). The methods of the present invention render the neuropathic pain more manageable even if it does not eliminate it (i.e., improves Quality of life).

Standard tests, well-known to the skilled person, am available in the art for assessing whether pain, in particular (chronic) neuropathic pain has been treated using a particular peptide of the invention. For example, the assessment of pain sensitivity in a subject has been standardized using quantitative sensory testing (pinpriks, pressure algometer, von Frey filaments, touch, pinching, or light pressure with the finger) or using a pain rating scale.

### Subject

In the context of the present invention, the subject or patient is an animal, preferably a mammal. In a particular aspect, the subject is a domestic animal, such as for example a horse, a dog, a cat, a cow, etc. In another particular and preferred aspect, the subject is a human-being.

The subject having chronic pain originating from a neuropathic pain may suffer from a disease classically associated with such neuropathic pain, such as for example fibromyalgia, complex regional pain syndrome, postherpetic neuralgia, Ehlers-Danlos syndrome and erythromelalgia.

In a particular aspect, the subject has fibromyalgia (FM). Fibromyalgia is a syndrome characterized by chronic musculoskeletal pain (Siracusa et al., 2021). FM is caused by a central sensitization phenomenon characterized by the dysfunction of neuro-circuits, which involves the perception, transmission and processing of afferent nociceptive stimuli, with the prevalent manifestation of pain at the level of the locomotor system. The main symptoms of this disease are muscle stiffness, joint stiffness, insomnia, fatigue, mood disorders, cognitive dysfunction, anxiety, depression, general sensitivity and the inability to carry out normal daily activities. FM can also be associated with specific diseases, such as an infection, diabetes, a rheumatic disease and/or a psychiatric or neurological disorder.

In a particular aspect, inventors herein describe the use of peptide(s), nucleic acid(s), expression cassette(s), vector(s), cell(s), and corresponding sets thereof (i.e., sets of such peptides, nucleic acids, expression cassettes, vectors or cells) as herein described, or composition comprising such peptides, nucleic acids, expression cassettes, vectors, cells or sets, as herein described, for preventing or treating fibromyalgia (FM).

In another aspect, the subject suffers from complex regional pain syndrome (CRPS). Complex regional pain syndrome is a chronic neurological condition involving the limbs that is characterized by severe pain along with sensory, autonomic, motor and trophic impairment (Goh et al., 2017). This condition may be induced by surgery, trauma or minor injury and has a varying course, ranging from mild and self-limiting, to chronic disease, which impairs activities of daily living and health-related quality of life. CRPS can be classified into two types: CRPS types I and II that are characterized by the absence or presence of identifiable nerve injury. CRPS type I is a syndrome that usually develops after an initiating noxious event, is not limited to the distribution of a single peripheral nerve, and is disproportionate to the inciting event. It is associated with oedema, changes in skin blood flow, abnormal sudomotor activity in the region of the pain, allodynia and hyperalgesia and commonly involves the distal aspect of the affected extremity or with a distal to proximal gradient. CRPS type II can be defined as a burning pain, allodynia and hyperpathia occurring in a region of the limb after partial injury of a nerve or one of its major branches innervating that region.

In a particular aspect, inventors herein describe the use of peptide(s), nucleic acid(s), expression cassette(s), vector(s), cell(s), and corresponding sets thereof (i.e., sets of such peptides, nucleic acids, expression cassettes, vectors or cells) as herein described, or composition comprising such peptides, nucleic acids, expression cassettes, vectors, cells or sets, as herein described, for preventing or treating complex regional pain syndrome (CRPS).

In still another aspect, the subject has postherpetic neuralgia (PHN). Postherpetic neuralgia is the most common complication of herpes zoster (HZ), an infection caused by the reactivation of dormant varicella zoster virus in the sensory ganglia (Ngo et al., 2020). It is characterized by a localized blistering rash and pain along the associated dermatome. PHN is defined as lingering pain for at least 90 days after the initial onset of HZ rash, and it significantly reduces the quality-of-life of affected patients. PHN is subcategorized into irritable nociceptor and deafferentation models. During the reactivation of VZV, the virus replicates and spreads from the dorsal root ganglion to its respective periphery. The propagation elicits an immune response and inflammation that damages the peripheral nerve. This damage decreases the neuron's inhibition of pain, lowering the threshold for depolarization of pain signals. This results in painful perception in response to non-painful stimuli, a process called peripheral sensitization. Repeated activation of subtype C-nociceptors also causes a heightened state of excitation in the dorsal horn. Direct viral damage by HZ weakens the descending inhibitory pain pathway, leading to a chronic activation of second-order neurons in the dorsal horn. Furthermore, loss of inhibitory gamma aminobutyric acid (GABA) producing interneurons in the dorsal horn has been reported in HZ patients with PHN when compared to HZ patients without PHN. These factors amplify all subsequent responses from the afferent input in a process called central sensitization. In PHN, this process is accompanied by the anatomical reorganization of low-threshold mechanoreceptive afferents, called Aβ-fibers, that normally relay harmless tactile stimuli to the central nervous system. When viral damage leads to the loss of C-nociceptors in the periphery, these fibers connect with second-order neurons that were originally wired to the C-nociceptor afferents in a compensatory manner. This process is called deafferentation, and patients suffering of allodynia exhibit a severe loss of sensory function.

In a particular aspect, inventors herein describe the use of peptide(s), nucleic acid(s), expression cassette(s), vector(s), cell(s), and corresponding sets thereof (i.e., sets of such peptides, nucleic acids, expression cassettes, vectors or cells) as herein described, or composition comprising such peptides, nucleic acids, expression cassettes, vectors, cells or sets, as herein described, for preventing or treating postherpetic neuralgia (PHN).

In still another aspect, the subject suffers from erythromelalgia (EM). Erythromelalgia is an infrequent episodic aerosyndrome affecting mainly both lower limbs bilaterally and symmetrically, or unilaterally, with the classic triad of erythema, warmth and burning pain (Maria Bibiana Leroux, 2018). EM is classified along with the chronic painful syndromes. Primary EM is an autosomal dominant inherited disorder encoded by OMIN (Online Mendelian Inheritance in Man) as #133020. It is associated with an alteration on the α subunit protein of the sodium channel type 9 (SCN9A), affecting the Nav1.7 channel that is expressed mainly in dorsal root ganglia and the sympathetic ganglia neurons. Secondary EM is associated with myeloproliferative diseases, paraneoplasias, autoimmune diseases, contact with a toxin and infections.

In a particular aspect, inventors herein describe the use of peptide(s), nucleic acid(s), expression cassette(s), vector(s), cell(s), and corresponding sets thereof (i.e., sets of such peptides, nucleic acids, expression cassettes, vectors or cells) as herein described, or composition comprising such peptides, nucleic acids, expression cassettes, vectors, cells or sets, as herein described, for preventing or treating erythromelalgia (EM).

In a particular aspect, inventors herein describe the use of peptide(s), nucleic acid(s), expression cassettes, vector(s), cell(s), and corresponding sets thereof (i.e., sets of such peptides, nucleic acids, expression cassettes, vectors or cells) as herein described, or composition comprising such peptides, nucleic acids, expression cassettes, vectors, cells or sets, as herein described, for preventing or treating any pain-associated condition wherein the signal transduced by nociceptors or interneurons is impaired.

Inventors previously established that the absence, a reduced or insufficient expression or non-functional expression of Myosin IA (Myola) in a subject when compared to the expression observed in a Myo1a^{+/+} reference subject expressing a functional Myola, predisposes the subject to develop an injury-induced chronic mechanical pain and/or an inflammatory-induced chronic thermal pain (WO2017153424).

In still another aspect, the subject is a subject having one or two mutated allele in the myo1A gene. By “mutated” allele is meant a substitution, deletion or insertion in the nucleic acid of myo1A gene (in a coding or non-coding region) that alter the expression or level of expression of Myo1a. The mutation can affect one or more nucleobases. The substitution can a Single Nucleotide Polymorphism (SNP).

In a particular aspect, inventors herein describe the use of peptide(s), nucleic acid(s), expression cassette(s), vector(s), cell(s), and corresponding sets thereof (i.e., sets of such peptides, nucleic acids, expression cassettes, vectors or cells) as herein described, or composition comprising such peptides, nucleic acids, expression cassettes, vectors, cells or sets, as herein described, for preventing or treating an injury-induced chronic mechanical pain and/or an inflammatory-induced chronic thermal pain in such a subject.

### Doses

The product according to the invention, in particular the peptides, set of peptides, or composition comprising such a set or allowing its expression, is preferably administered directly to the subject in a therapeutically effective amount. The terms “therapeutically effective amount” refer to the amount of peptides of interest, in particular of peptides of SEQ ID NO: 1 and SEQ ID NO: 2 or of functional variant(s) thereof, required to treat, ameliorate, or prevent pain in a subject. The therapeutically effective amount can be estimated initially either in cell culture assays, or in animal models. An animal model may also be used to determine the appropriate concentration range and route of administration of the (set of) peptides. Such information can then be used to determine useful doses and routes for administration in humans.

The dosage of the peptide used in the methods herein described may vary depending on the general health condition, age, gender and weight of the subject, the nature and severity/intensity of pain, time, frequency and duration of administration, the particular peptides being used, drug combination(s), reaction sensitivities, and tolerance/response to therapy. This suitable effective dosage can be determined by routine experimentation and is under the judgement of the clinician.

The dose may be administered in the bolus form or may be divided into several portions (/units), which are administered separately along the day. In other words, the treatment can be a single dose schedule or a multiple doses schedule. The effective dose may be administered for a period of days, weeks, months or years.

To obtain suitable antalgic or analgesic effect, the effective total dose of the peptides or variants thereof present in a set of peptides as herein described by inventors, or in a composition comprising such a set of peptides, typically of both the peptides of SEQ ID NO: 1 and the peptide of SEQ ID NO: 2, is between 1 μg and 10 g, preferably 1 μg and 100 mg, more preferably between 25 μg and 10 mg, even more preferably between 2 mg and 10 mg per unit dose.

In a particular aspect of the invention, the set of peptides or composition comprising such a set of peptides is administered at a dose between 1 μg per kg of body weight per 24 hours (i.e., 1 μg/Kg/day) and 100 ng/kg/day, preferably between 2.5 μg/kg/day and 0.6 mg/kg/day, for a mammal.

When the subject is a human being, the effective dose of the peptide or variant thereof as herein described by inventors administered to the subject is preferably between 2.5 μg/kg/day and 0.6 mg/kg/day, preferably between 5 μg or 10 μg/kg/day and 0.5 mg/kg/day, more preferably between 50 μg or 75 μg/kg/day and 0.3 mg/kg/day if the subject is a human subject.

In a particular aspect of the invention, the set of peptides or composition comprising such a set of peptides is administered at a dose wherein the peptide of SEQ ID NO: 1 and the peptide of SEQ ID NO: 2 are at an equivalent molarity.

In a more particular aspect of the invention, the set of peptides or composition comprising such a set of peptides is administered at a dose comprising between 50 μg/kg and 150 μg/kg of the peptide of SEQ ID NO: 1 and between 100 μg/kg and 250 μg/kg of the peptide of SEQ ID NO: 2.

Delivery of a product as herein described, in particular of peptides or of a composition as herein described, to the subject in need thereof, may be accomplished by any route.

The product herein described can be administered for example intramuscularly, intravenously, intraperitoneally, orally (per os), anally, cutaneously, subcutaneously, topically, dermically, transdermically or intrathecally. Preferably, the product herein described is administered subcutaneously, orally or intravenously, even more preferably subcutaneously or orally.

As shown in the experimental part, the inventors demonstrate that the set of peptides comprising the peptide of SEQ ID NO: 1 and the peptide of SEQ ID NO: 2 has antalgic effect when administered subcutaneously to treat pain, in particular neuropathic pain. Surprisingly, the antalgic effect is also observed when the set of peptides comprising the peptide of SEQ ID NO: 1 and the peptide of SEQ ID NO: 2 is administered orally (per os) or intravenously. Even more surprisingly and advantageously, the set of peptides of the invention shows better antalgic effect than the TAFA-4 (full length) protein when administered per os or subcutaneously which makes it more convenient and suitable for being administered to the patient than said full length protein.

Advantageously, the set of two peptides of the invention, or a composition comprising such a set, while being much easier (and consequently cheaper) to produce than the full length TAFA-4 protein, induces a higher antalgic activity/effect in comparison with the latter, when administered subcutaneously. Even more surprisingly, when it comes to oral (per os) administration, the set of peptides of the invention shows better antalgic effect in comparison with the TAFA-4 (full length) protein which makes it more convenient and suitable for being administered to the patient than said TAFA-4 full length protein. Intravenous injection of the set of two peptides advantageously offers a third route of administration for treating neuropathic pain.

### Kits

Each of the product herein described, for example each of the products herein described, preferably each peptides, each set of peptides, or each composition herein described, may be part of a kit.

A typical kit of the invention comprises at least two products as herein described selected from a peptide or variant thereof, a nucleic acid, an expression cassette, a vector, a cell, a set of peptides, a set of nucleic acids, a set of expression cassettes, a set of vectors, a set of cells, and a composition comprising such peptides, nucleic acids, expression cassettes, vectors, cells or sets, and optionally at least one additional distinct active compound efficient against pain. In a particular aspect, the kit further comprises written instructions for using the kit.

The kit is preferably a kit-of-parts comprising at least two parts, for example two distinct containers, wherein a first part comprises a peptide, a nucleic acid, an expression cassette, a vector, a cell, a set of peptides, nucleic acids, expression cassettes, vectors, or cells, or a composition as disclosed herein, and the second part comprises at least one additional distinct active compound efficient against pain. Preferably, the active compound efficient against pain is a steroidal anti-inflammatory drug (SAID), a nonsteroidal anti-inflammatory drug (NSAID) or an opioid drug as herein disclosed.

In a particular aspect, the kit comprises i) a first peptide of SEQ ID NO: 1, a peptide having at least 90% identity to SEQ ID NO: 1, or a nucleic acid sequence encoding such a peptide, ii) a second peptide of SEQ ID NO: 2, a peptide having at least 90% identity to SEQ ID NO: 2, or a nucleic acid sequence encoding such a peptide, the first and second peptides, or the nucleic acids encoding said peptides, being in distinct containers, and, optionally, iii) at least one additional distinct active compound efficient against pain, and/or iv) written instructions for using the kit.

In one aspect, the peptide, the nucleic acid, the expression cassette, the vector, the cell, the set or the composition of the kit is in a form adapted for intramuscular, intravenous, intraperitoneal, oral (per os), anal, cutaneous, subcutaneous, topical, dermical, transdermical or intrathecal routes, preferably subcutaneous or oral route, even more preferably oral route.

In another aspect, the at least one additional distinct active compound efficient against pain is in a form adapted for intramuscular, intravenous, intraperitoneal, oral (per os), anal, cutaneous, subcutaneous, topical, dermical, transdermical or intrathecal routes.

Depending on the nature, the origin, the intensity of pain to be treated, and also depending on the nature of the peptide, nucleic acid, expression cassette, vector, cell, set or content of the composition and on the nature of the at least one additional distinct active compound efficient against pain, said products are co-administered or not, simultaneously/concomitantly or sequentially.

The present invention also relates to the use in vivo, ex vivo or in vitro of a kit of the invention for preventing or treating pain as described hereinabove, for example chronic pain, neuropathic pain, postoperative pain, inflammatory pain, hyperalgesia or allodynia. The kit of the invention may also be used in prevention or treatment of acute or subacute pain.

Also herein disclosed is the use of a kit of the invention for the manufacture of a pharmaceutical composition, drug or medicament for preventing or treating pain as described hereinabove in a subject in need thereof, for example chronic pain, neuropathic pain, postoperative pain, inflammatory pain, hyperalgesia or allodynia. The kit of the invention may also be used for the manufacture of a pharmaceutical composition, drug or medicament for prevention or treatment acute or subacute pain in a subject in need thereof.

### Research Tools

In a particular aspect, the products of the present invention may also be used in the context of research.

The present invention also includes the use of peptide(s) or a variant(s) thereof as herein described, in particular of a set of peptides, or of any other product herein above described, as a research tool.

The nucleic acids encoding peptide(s) or a variant(s) thereof as herein described, the expression cassettes or vectors permitting their expression, or the cell comprising such nucleic acid(s) or modified using such expression cassettes or vector(s) are typically used to express or modulate in vitro or ex vivo the expression of peptides of interest, in particular of a peptide of SEQ ID NO: 1 or a functional variant thereof having at least 90% identity to SEQ ID NO: 1 and/or of a peptide of SEQ ID NO: 2 or a functional variant thereof having at least 90% identity to SEQ ID NO: 2.

These tools may be used for studying pain or for modulating neuronal excitability in a biological tissue or in cell cultures for example to study mechanically and/or chemically induced pain nociceptive signals.

The present invention also relates to the use of nucleic acid(s) encoding a peptide of SEQ ID NO: 1 or a peptide having at least 90% identity to SEQ ID NO: 1 and a peptide of SEQ ID NO: 2 or a peptide having at least 90% identity to SEQ ID NO: 2, or of expression cassette(s) or vector(s) permitting their expression, to express or modulate the (levels of) expression of the peptide of SEQ ID NO: 1 or of a peptide having at least 90% identity to SEQ ID NO: 1 and of the peptide of SEQ ID NO: 2 or of a peptide having at least 90% identity to SEQ ID NO: 2, in a biological tissue or a culture of cells.

Nucleic acid molecules herein described may also be used to create transgenic animals. This may be done locally by modification of somatic cells or through germ line therapy for the germinal cells to incorporate heritable modifications. The present invention therefore also relates to a transgenic organism (e.g. animal) comprising (i.e. containing) nucleic acid(s) of SEQ ID NO: 3 and/or SEQ ID NO: 4 or variants thereof; an expression cassette or vector comprising a nucleic acid of SEQ ID NO: 3 and/or SEQ ID NO:4 or a variant thereof; or expressing a peptide of SEQ ID NO: 1 or a peptide having at least 90% identity to SEQ ID NO: 1 and/or a peptide of SEQ ID NO: 2 or a peptide having at least 90% identity to SEQ ID NO: 2. The invention therefore also relates to a host cell or transgenic organism, in particular transgenic animal, containing nucleic acid sequence(s) encoding, or expression cassette(s) or vector(s) permitting the expression of, any set of (at least two) peptides as herein described.

The following examples are provided in order to demonstrate and further illustrate certain preferred aspects of the present invention and are not to be construed as limiting the scope thereof.

### EXAMPLES

### Materials and Methods

### Peptides Synthesis

All peptides were synthesized at 200 μmole scale using Fmoc-SPPS on a Symphony X instrument (Protein Technologies. Inc., USA). Synthesis-grade Fmoc-AA reagents were purchased from Iris Biotech. Specific L-pseudoproline dipeptides was used to facilitate the peptide syntheses. HATU, 2-chlorotrityl chloride resin (200-400 mesh, 1.2 mmol/g), RINK amide resin (200-400 mesh, 0.42 mmol/g) were purchased from IRIS Biotech. DMF, DCM, methanol, diethyl ether, and HPLC-grade acetonitrile were from purchased from Aldrich. The standard deprotection-coupling cycle for each residue consisted of six steps:
1. Wash with 5 mL of DMF (3×30 sec)
2. Deprotect Fmoc group with 5 mL of 20% piperidine in DMF (3×3 min)
3. Wash with 5 mL of DMF (3×30 sec)
4. Couple Fmoc-AA (2×60 min)
a. 5 mL of Fmoc-AA dissolved at 200 mM in DMF
b. 2 mL of HATU dissolved at 500 mM in NMP
c. 2 mL of iPr₂NEt dissolved at 1 M in NMP
5. Capping with 5 mL of 10% Ac₂O in DMF (7 min)
6. Wash with 5 mL of DMF (3×30 sec)

Upon completion of the synthesis, peptide resins weir washed 3× with DMF and 3× with DCM. Peptide resins were then cleaved with one of three TFA cleavage cocktails (per 100 μmole scale synthesis):
35 mL TFA, 1000 μL TIS, 2000 μL water, 1000 mg DTT

After 3 h of TFA cleavage, the cleavage solution was filtered from the resin, and peptides were precipitated by addition to 40 mL of ice-cold ether. After >1 h at −20° C., ether solutions were centrifuged at 3500 RCF, and the supernatants were decanted. Pellets were washed twice more with ether, and then dried for >3 h in vacuum desiccator prior to dissolution, analytical characterization, and purification.

### General Methods for Preparation of Peptide Resins Hydrazide

0.35 mmol Fmoc-NH—NH₂ was dissolved in 30 mL DCM, and then 1.5 mmol DIPEA was added to the solution. This solution was next added to 1 g of resin and mixed for 2 h at room temperature. Unreacted groups were capped with 17:2:1 DCM:MeOH:DIPEA for 30 min. Fmoc-AA-resin was then washed 3× with DMF, 3× with DCM and vacuum dried for >20 min. The loading was determined by the titration of the Fmoc group deprotection (UV, 301 nm).

### Analysis and Purifications

The peptides were analyzed by UPLC and ESI-MS mass spectrometry. The instruments were equipped with BEH C18 (WATERS), 150*2.1 mm (150×2.1 mm) (flow rate: 0.6 mL/min). Solvents A and B were 0.1% TFA in water and 0.1% TFA in acetonitrile.

Purifications of crude peptides were performed with a preparative reversed phase HPLC (Waters Delta Prep 4000) system using a reversed phase column (Vydac Denali prep C-18, 10 μm, 120 Å, 50×300 mm) and an appropriate gradient of increasing concentration of buffer B in buffer A (flow rate of 80 mL/min). The fractions containing the purified target peptide were identified by UV measurement (Waters 2489 UV/Visible detector) at 214 nm and selected fractions were then combined and lyophilized.

(peptide F1 - SEQ ID NO: 1)
Synthesis of Ac-CFPGQVAGTTRAQPSCVEASIVIQKWW-NHNH₂;

Peptide F1 (Ac-CFPGQVAGTTRAQPSCVEASIVIQKWW-NHNH2) was prepared by standard Fmoc-SPPS as described above using hydrazide resin, Standard peptide cleavage conditions (described above) was employed following.

After standard cleavage procedure, F1 crude peptide was dissolved in water and purified on a RP-HPLC. The fractions containing the purified target peptide were identified and selected fractions were then combined and lyophilized.

(peptide F2 - SEQ ID NO: 2)
Synthesis of CHMNPCLEGEDCKVLPDYSGWSCSSGNKVKTTKVTR-
NH₂:

Peptide F2 (CHMNPCLEGEDCKVLPDYSGWSCSSGNKVKTTKVTR-NH₂) was prepared by standard Fmoc-SPPS as described above using RINK amide resin.

After standard cleavage procedure, F2 crude peptide was dissolved in water and purified on a RP-HPLC. The fractions containing the purified target peptide were identified and selected fractions were then combined and lyophilized.

### Mice

C57/Bl6J mice (8 to 12 weeks of age) were bought from Charles River Laboratories. Mice of both sexes were used for all experiments. As no differences were noted between males and females, the data for the two sexes were then combined. Mice were maintained under standard housing conditions (22° C., 40% humidity, 12 h light cycles, and free access to food and water). Particular efforts were made to minimize the number of mice used in this study, and the stress and suffering to which they were subjected. All experiments were conducted in line with the European guidelines for the care and use of laboratory animals (Council Directive 86/609/EEC). All experimental procedures were approved by an independent ethics committee for animal experimentation (APAFIS), as required by the French law and in accordance with the relevant institutional regulations of French legislation on animal experimentation. All experiments were performed in accordance with the ARRIVE guidelines.

### Pain Model 1: Spared Nerve Injury (SN) Model—Neuropathic Pain.

The Spared Nerve Injury (SNI) model, developed by Decosterd and Woolf, 2000; Pain, Vol. 87, p 149-158 was used. It was used as a neuropathic pain model. The SNI model consists in the transection of tibial branches and of the common peroneal nerve of the sciatic nerve: the sural nerve remaining intact. The latter then develops signs of neuropathic pains with substantial mechanical allodynia. The SNI model has many advantages:
neuropathic pain is persistent. This allows to grasp habituation phenomena upon repeated injections of the peptide.
the generated pain is robust.
the model is very reproducible.

Mice were anesthetized with ketamine (100 mg/kg IP) and xylazine (10 mg/kg IP) and the left sciatic nerve was exposed under aseptic conditions. The distal trifurcation of the sciatic nerve was identified and the tibial and common peroneal branches were ligated with polypropylene nonabsorbable 6-0 sutures (Ethicon); 1 mm was cut out, leaving the sural branch intact. The wound was closed with sutures, and the animals were allowed to recover and returned to their cages.

### Pain Model 2: Paw Incision—Postoperative Pain.

Paw incision surgery was performed as described by Brennan and co-workers (1999) (Brennan, 1999). Mice were anesthetized with ketamine (100 mg/kg IP) and xylazine (10 mg/kg IP) and a longitudinal incision was made through the skin and fascia of the right hind paw. Forceps were used to elevate the flexor digitorum brevis muscle longitudinally and an incision was made through the muscle with a scalpel, to cut it into two halves. The wound was closed with sutures, and the animals were allowed to recover and returned to their cages. The paw incision was used as postoperative pain model.

Pain model 3: Carrageenan Injection—Inflammatory Pain.

Inventors injected 20 μl of 1% λ-carrageenan (Sigma-Aldrich, 22049-5G-F) in 1×PBS into the plantar surface of the left hind paw of the mouse with a Hamilton syringe. The carrageenan injection was used as an inflammatory pain model.

### Pain Model 4: Chronic Constriction Injury (CCI) Model—Neuropathic

CCI neuropathic pain model: Chronic Constriction Injury (CCI) was performed as described previously by Bennett and Xie 1988 (A peripheral mononeuropathy in rats that produces disorders of pain sensation like those in man. Pain vol 33: pp 87-107). Briefly, unilateral peripheral mononeuropathy was induced in mice anaesthetized with Ketamine/Xylasine (respectively 100 mg/kg and 10 mg/kg ip) with two ligatures (6-0 Monocryl, Ethicon) tied loosely (with about 1 mm spacing) around the common sciatic nerve. The nerve was constricted to a barely discernible degree, so that circulation through the epineural vasculature was not interrupted.

### Pain Model 5: Capsaicin Inflammatory Pain.

Capsaicin (Sigma) stock solution (dissolved in absolute ethanol 16.7 mg/mL) was diluted in NaCl 0.9% at 0.25 mg/mL, then 10 μL were injected into the plantar surface of the left hind paw of the mouse using a Hamilton syringe and a 26G needle.

### Pain Model 6: Osteoarthritis Pain Model (Mono-Iodoacetate Model).

The MIA model is a standard model for joint disruption in osteoarthritis in rodents. Briefly, mice were anesthetized with ketamine (100 mg/kg IP) and xylazine (10 mg/kg IP), followed by injection of 1 mg/10 μL of MIA (mono-iodoacetate, Sigma) in NaCl 0.9% into the synovial fluid of the knee of left hind paw of the mouse using a Hamilton syringe and a 26G needle as described by Pitcher et al. (2016).

### Pain Model 7: Ultraviolet-Burn Pain Model

Mice were anesthetized with ketamine (100 mg/kg IP) and xylazine (10 mg/kg IP) and completely covered except for the plantar side of one hind paw. The paw was irradiated using a UV lamp (VL-215.MC Vilmer) with a wavelength of 312 nm at a distance of 20 cm for 60 minutes. Once awake, the animals are returned to their cages.

### Pain Model 8: Freeze-Burn Pan Model

Mice were anesthetized with ketamine (100 mg/kg IP) and xylazine (10 mg/kg IP). Hind paw was exposed to cold via a copper tube put in dry ice and in contact through a NaCl soaked piece of filter paper for 40 seconds. Once awake, the animals are returned to their cages.

### Von Frey's Test

The Von Frey's uses Von Frey (VF) hair or fibers, which are small pieces of nylon rod, approximately 50 mm in length to test a rodent's sensitivity to a mechanical stimulus. In this test, the animal stands on an elevated mesh platform, and the Von Frey hairs are inserted through the mesh to poke the animal's hindpaw. Normal reactions for the animal include withdrawing or licking or shaking the paw. The “up-down” Von Frey method is used to determine the mechanical force required to elicit a paw withdrawal response in 50% of animals. Here, mice were placed in plastic chambers on a wire mesh grid and stimulated with von Frey filaments (Vivotech) by the “up-down” method (45) starting with a 1 g filament, and using 0.04 and 4 g filaments as the cutoffs.

### Statistical Analysis

The results are expressed as means±SEM. Statistical analyses were performed with Prism 7 (Graphpad Software, La Jolla, CA, USA).

### Results

### Example 1: Pain Model 1: Spared Nerve Injury (SNI Model—Neuropathic Pain

### 1.1 Comparison of the Antalgic Effects of the TAFA-4 Full Length Protein (“TAFA-4 FL”) and of the Set of Two Peptides of SEO ID NO:1 and of SEO ID NO: 2 (“Peptides 1+2”) in the Context of a Subcutaneous Administration.

The purpose of these experiments was to assess the antalgic effect of a set of two peptides according to the invention (this set being herein identified as “Peptides 1+2” or “Peptides F1+F2”) by subcutaneous injection in the Spared Nerve Injury (SNI) model of neuropathic pain, and to compare this effect with that obtained with the TAFA-4 full length protein (“TAFA-4 FL”).

The experiments were conducted on eight-week-old male WT C57Bl6 mice. Four groups of 6 to 12 mice were used. Peptides of SEQ ID NO: 1 and of SEQ ID NO: 2 were resuspended in 0.9% NaCl at [0.6 mg/mL]. The two peptides were diluted to obtain equivalent molarity (12.8 μg/ml for the peptide of SEQ ID NO: 1 and 17.2 μg/ml for the peptide of SEQ ID NO: 2) in order for it to be used at a final total concentration of “peptides 1+2” of 0.3 mg/kg in this study. Similarly, TAFA-4 FL was resuspended in 0.9% NaCl, diluted and used at a final concentration of 0.3 mg/kg.

0.9% NaCl solution is used as a negative control (vehicle) and 3 mg/kg of pregabalin, a well-known drug used to treat neuropathic pain, is used as a positive control.

After having measured the base threshold (“baseline”) of mice with Von Frey (VF) filaments by the up/down method [showing the mechanical force required (expressed as its weight equivalent in grams) to elicit a paw withdrawal response in 50% of mice as a function of time under different condition], the SNI model is set up. The mice are anesthetized; ligature of the tibial nerve and of the fibular nerve is performed; and these two nerves are then severed. The sural nerve left intact develops neuropathy quite rapidly. The occurrence of neuropathy is ascertained after 3 days post-surgery. A decrease in the response threshold to Von Frey filaments of the ipsilateral paw is thereby observed.

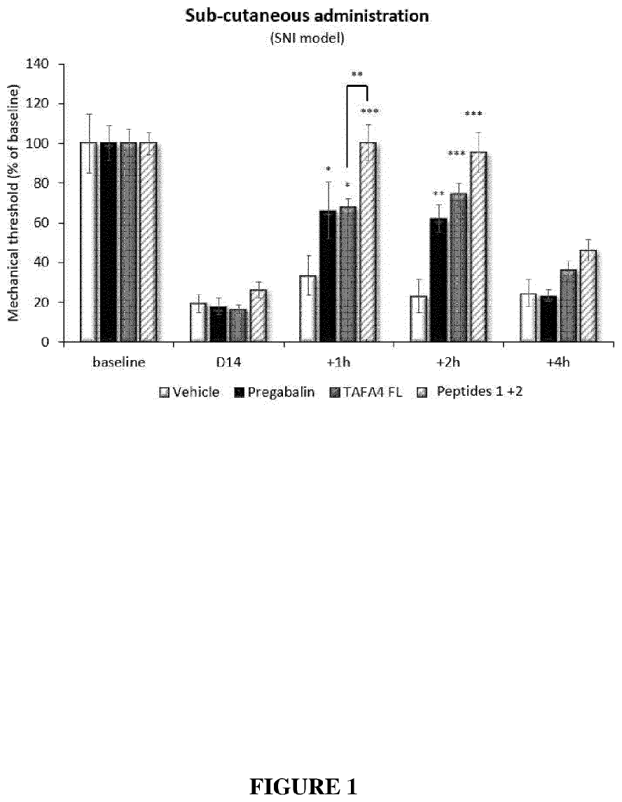

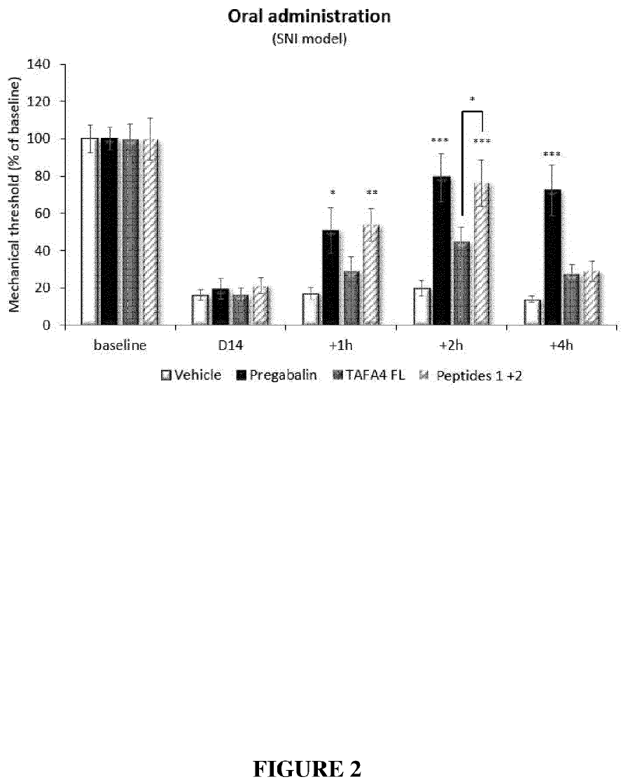

14 days after surgery, the response threshold is again measured (cf. “1314” on FIG. 1). 100 μl/10 g of each of the solutions comprising respectively the set of two peptides, the TAFA-4 full length protein, the vehicle or pregabalin are then blind-injected subcutaneously to mice by the experimenter.

The response threshold is measured after 1 hour, 2 hours and then 4 hours after administration/injection.

At day 14 post-SNI, all mice exhibited a drastic decrease in the mechanical thresholds, illustrative of a strong mechanical hypersensitivity as compared to the baseline threshold (cf. FIG. 1). Subcutaneous administration of the vehicle had no effect. Surprisingly, at one hour post administration, the antalgic effect of the set/composition of two peptides of SEQ ID NO: 1 and of SEQ ID NO: 2 (“Peptide 1+2”) was maximum and was significantly higher than that of the TAFA-4 full length protein (“TAFA4 FL”) (with p value<0.01), and also higher than that of pregabalin. Statistical analysis shows strong significant result (with p value<0.001) at 1 hour and 2 hours for the set/composition of two peptides in comparison with negative control (vehicle).

1.2 Comparison of the Antalgic Effect of the TAFA-4 Full Length Protein (“TAFA-4 FL”) and of the Set of Two Peptides of SEO ID NO: 1 and of SEO ID NO: 2 (“Peptides 1+2”), in the Context of a Per Os Administration.

The protocol described in 1.1 was used except that the administrations of the set of two peptides, of the TAFA-4 FL, of the vehicle and of pregabalin was done 14 days after surgery using per os administrations.

For per os administration, the two peptides of SEQ ID NO: 1 and of SEQ ID NO: 2 were diluted at a total concentration of 30 μg/mL in a solution of 1% w/v Hydroxypropylmethylcellulose (Sigma-aldrich #423238, batch MKCD3665), 0.5% v/v Tween 80 (Euromedex #2002-A, batch 100412/16S407), referred herein below as HPMC solution or “vehicle”. Similar protocol was applied for TAFA-4 FL.

Vehicle, TAFA-4 FL solution and the solution comprising the set of two peptides were orally administered using two gavage needles. Pregabalin (3 mg/kg) was administered per oral gavage (“per os”) by a different experimenter from that performing the VF measurements so that the latter worked blind for the treatment (n=8 for each treatment). Administrations were performed at 14 days after surgery.

At day 14 post-SNI, all mice exhibited a drastic decrease in the mechanical thresholds (FIG. 2).

Oral administration of the vehicle had no effect. The TAFA-4 full length protein did not show significant antalgic effect when administered orally (i.e. per os). Surprisingly, oral administration of the set/composition of two peptides of the invention induced a statistically significant reversal effect on mechanical threshold as soon as 1 hour post administration (p value<0.01), with a maximum effect occurring at 2 h post administration (p value<0.001). The antalgic effect of the set/composition of two peptides of the invention was similar to that of pregabalin at 1 hour and 2 hours post administration whereas it was significantly higher than that of the TAFA-4 full length protein (“TAFA4 FL”) at 2 hours post administration. From FIG. 2, it can be concluded that, when administered per os, the set/composition of two peptides of the invention has a much better antalgic effect than the TAFA-4 full length protein.

### 1.3 Antalgic Effect of a Set of Two Peptides of SEO ID NO: 1 and of SEO ID NO:2 (“Peptides 1+2”) Administered Intravenously

The protocol described in 1.1 was used except that the administration of the set of two peptides or of the vehicle was done 14 days after surgery using the intravenous route.

The peptides of SEQ ID NO: 1 and of SEQ ID NO: 2 were resuspended in 0.9% NaCl at a total concentration of 150 μg/mL.

As previously described, the Von Frey filament measurement with the up/down method is done for determining the baseline. Then, the SNI model is set into place. The response threshold fourteen days (DAY 14—D14) after surgery is measured to check for the occurrence of neuropathic pain. Then, blind injection of 20 μl/10 g of the solution comprising the set of two peptides at a total concentration of 300 μg/kg was slowly performed in the tail vein. The response threshold was measured after 1 hour, 2 hours, and then 4 hours, after intravenous administration. Vehicle (n=11) were also administered intravenously.

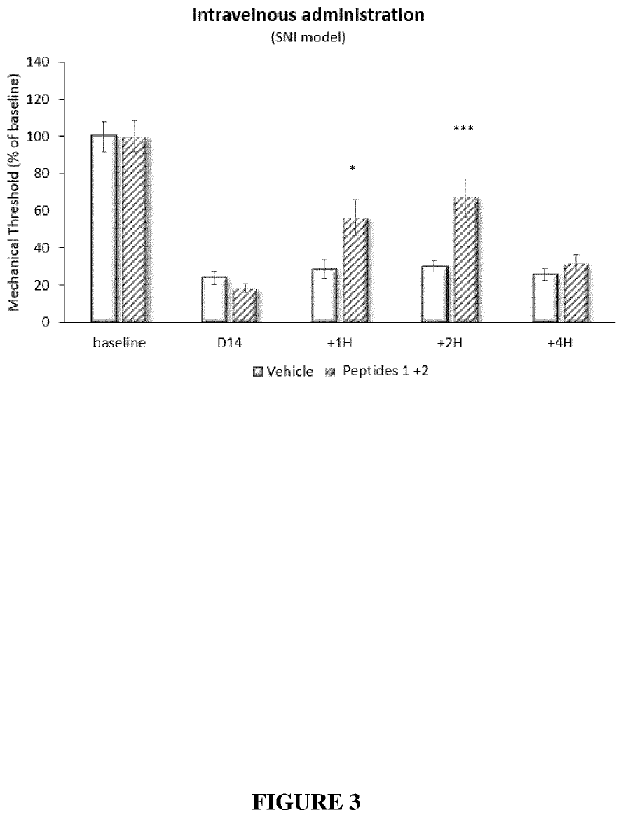

At day 14 post-SNI, all mice exhibit a drastic decrease in the mechanical thresholds, illustrative of a strong mechanical hypersensitivity, as compared to the baseline thresholds (FIG. 3). Intravenous administration of the vehicle had no effect. Surprisingly, a strong significant antalgic effect of the set of two peptides of SEQ ID NO: 1 and of SEQ ID NO: 2 (“Peptides 1+2”) was observed, with the maximum reversal effect on mechanical threshold observed at 2 h (p<0.001).

Altogether, the present experiment shows that the set of two peptides of the invention induced an antalgic effect by subcutaneous, per os and intravenous administration in the SNI model (neuropathic pain model). Advantageously, the very early (i.e., 1 h post administration) antalgic effect of the set of two peptides of the invention is more significant than that observed with the TAFA-4 (full length) protein when administered subcutaneously. Even more surprisingly, the set of two peptides of the invention shows better antalgic effect than the TAFA-4 (full length) protein when administered per os at 2 h post administration.

Inventor's results demonstrated the antalgic effect of the set of the two peptides of SEQ ID NO: 1 and of SEQ ID NO: 2 in a neuropathic pain model. Mechanical allodynia (decreased threshold of pain sensitivity) induced by this model can be reduced by the subcutaneous, intravenous or oral (per os) administration of the set of peptides of the invention.

Advantageously, the set of two peptides of the invention, or a composition comprising such a set, while being much easier (and consequently cheaper) to produce than the full length TAFA-4 protein, induces a higher antalgic activity/effect in comparison with the latter, when administered subcutaneously. Even more surprisingly, when it comes to oral (per os) administration, the set of peptides of the invention shows better antalgic effect comparison to the TAFA-4 (full length) protein which makes it more convenient and suitable for being administered to the patient than said TAFA-4 full length protein. Intravenous injection of the set of two peptides advantageously offers a third mute of administration for treating neuropathic pain.

### Example 2: Paw Incision—Postoperative Pain

The purpose of these experiments was to assess the antalgic effect of a set of two peptides of SEQ ID NO:1 and of SEQ ID NO:2 according to the invention (this set being herein identified as “Peptides 1+2”) by subcutaneous injection in the postoperative pain model (Brennan paw incision model).

The experiments were conducted on eight-week-old male and female mice WT C57Bl6. 12 males and 12 females for “Peptides 1+2”-treated group were used whereas 11 males and 11 females for vehicle group were used. Equimolar combination of Peptides of SEQ ID NO: 1 and of SEQ ID NO: 2 were resuspended in Ultra-Pure Water at [4 mg/mL]. The two peptides were diluted to obtain a final total concentration of “peptides 1+2” of 0.3 mg/kg in this study.

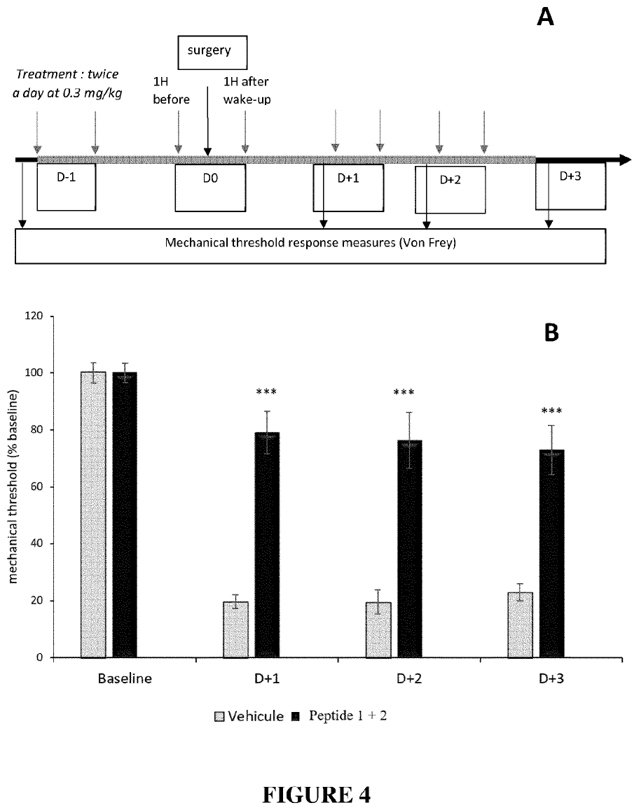

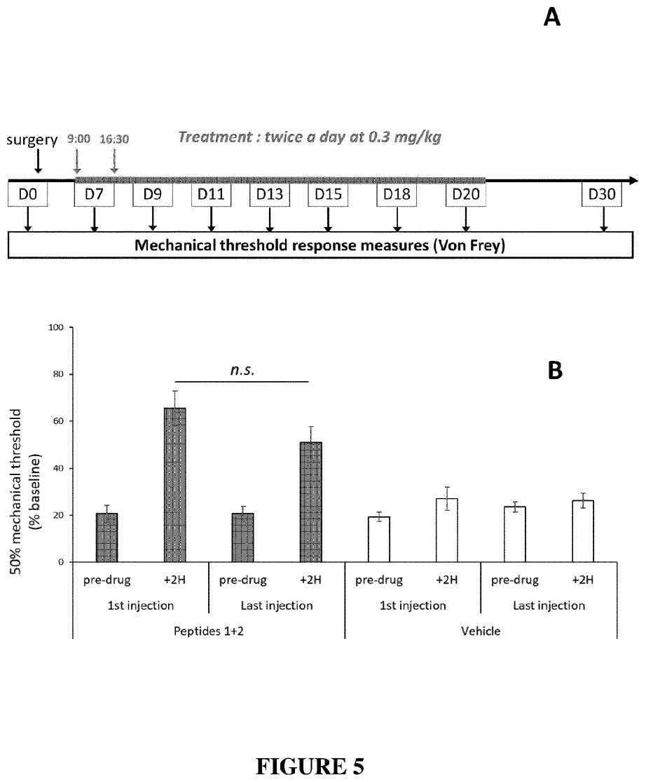

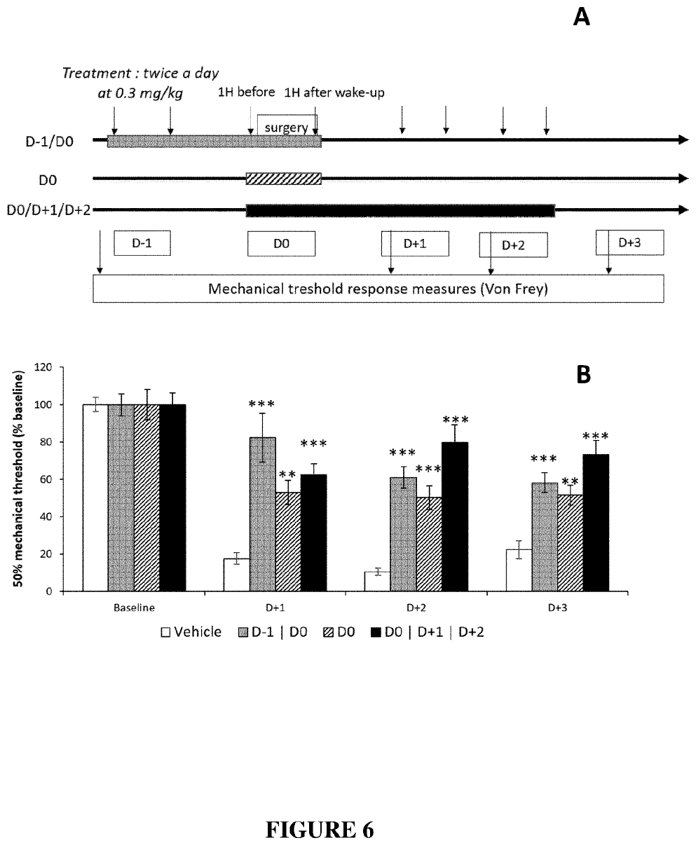

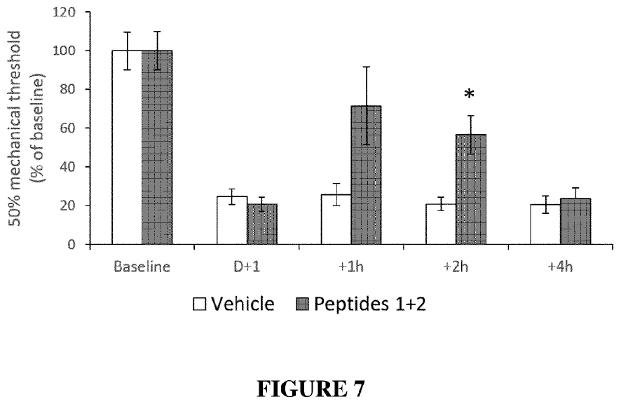

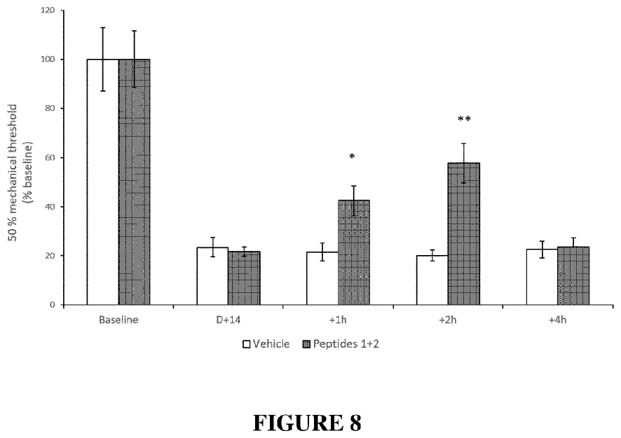

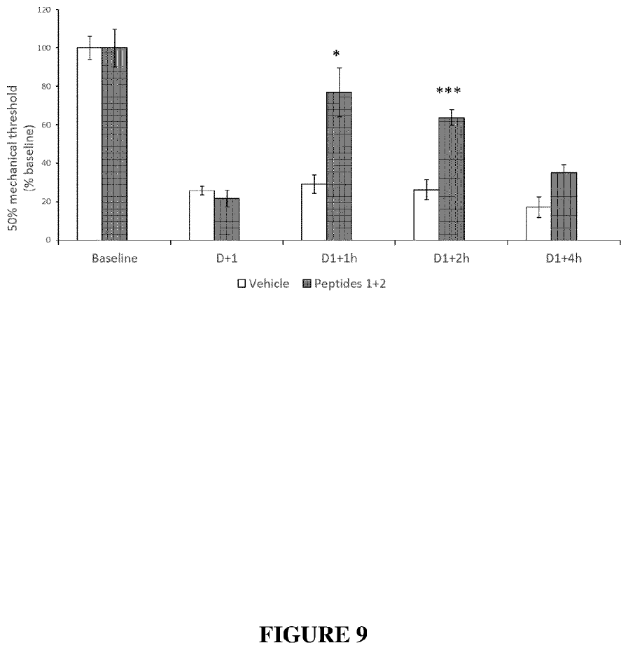

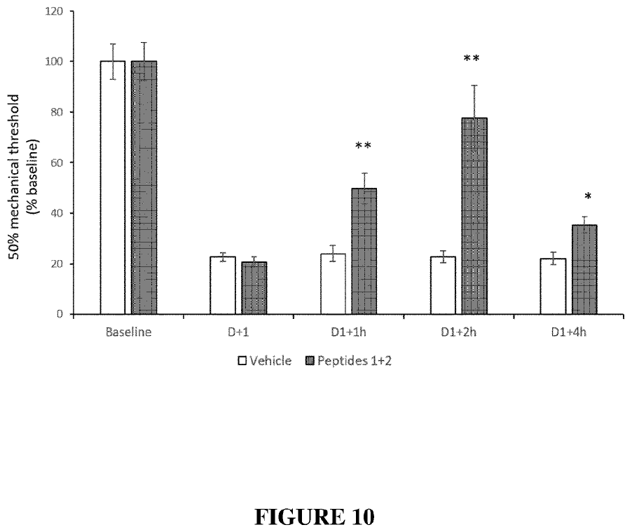

“Peptides 1+2” or a vehicle were subcutaneously administered twice a day at different time point: the day before surgery (D-1), the day of surgery (D0), and at D+1 and D+2 after surgery. The administrations at D0 were performed 1 hour before and 1 hour after surgery (and wake up), respectively. Mechanical threshold response measurements at D+1 (before injection at D+1), D+2 (before injection at D+2) and D+3 were performed (FIG. 4A).

After paw incision, mice treated with vehicle developed mechanical allodynia (see at D+1, D+2 and D+3 on FIG. 4B). On the contrary, mice treated with “Peptides 1+2” did not develop mechanical allodynia. In comparison with mice treated with vehicle, mice treated with “Peptides 1+2” present a statistically significant higher response threshold at D+1. D+2 and D+3 (FIG. 4B).

These results show that the set of peptides of the invention can be used as antalgic in a paw incision model (postoperative pain model).

### Example 3: Absence of Tolerance in Chronic Constriction Injury (CCI) Neuropathic Pain Model

The purpose of this experiment was to assess whether the subject treated with the set of peptides of SEQ ID NO:1 and of SEQ ID NO: 2 (“Peptides 1+2”) may induce a tolerance towards this set of peptides after repeated subcutaneous administration of the set of peptides.

The CCI model is put in place according to the protocol described above. Starting at D7 post-surgery, mice were subcutaneously injected with “Peptides 1+2” (0.3 mg/kg) twice a day for 14 consecutive days. Mechanical sensitivity is tested using Von Frey filaments with the up/down method before surgery and 7 days after to validate CCI-induced mechanical allodynia. A time course response was performed after the first administration. Then, every other day, antalgic effect of “Peptides 1+2” was tested. Then, after the last administration (D20), a complete time course response was performed (FIG. 5A). A last measure was performed at D+30 (10 days after the end of the treatment) to verify the correct recovery of mice.

The first subcutaneous injection of “Peptides 1+2” induced a strong increase in the response (i.e., paw withdrawal) threshold demonstrating the antalgic effect of “Peptides 1+2” (FIG. 5B—comparison between the response measured in “predrug” conditions vs. two hours after administration at first injection). Interestingly, the antalgic effect of “Peptides 1+2” remains constant for the entire duration of the treatment (14 days) without any decrease of efficacy, even at the last injection corresponding to D20 (FIG. 5B, non-significant difference between the response measured as the first injection in comparison with the response measured at the last injection). These results show that the set of peptides of the invention can be used repeatedly over a long period of time without inducing a tolerance in a subject.

### Example 4: Comparison of Different Posology of Treatment Postoperative Pain Model—Preventive Effect

The purpose of this experiment was to test different posology for treating a subject suffering from postoperative pain with the set of peptides of SEQ ID NO:1 and of SEQ ID NO:2 (“Peptides 1+2”) when administered subcutaneously in vivo in a postoperative pain model.

“Peptides 1+2” or a vehicle were administered subcutaneously twice a day according to three different protocols. “D-1/D0” protocol: the administration was performed the day before surgery (D-1), and the day of surgery (D0); “D0” protocol: the administration was performed the day of surgery (D0); “D0/D+1/D+2” protocol: the administration was performed the day before surgery (D-1), the day of surgery (D0), and at days D+1 and D+2 after surgery. The administrations at D0 were performed 1 hour before and 1 hour after surgery (and wake up), respectively. Mechanical threshold response measurements at D+1 and D+2 were performed before administration of the set of two peptides or vehicle (FIG. 6A).

After paw incision, mice treated with a vehicle developed mechanical allodynia (see at D+1, D+2 and D+3 on FIG. 6B). On the contrary, mice treated with “Peptides 1+2” did not develop mechanical allodynia regardless of the protocol used. Interestingly, mice treated with “D-1/D0” and “D0” protocols, which were not treated after surgery, didn't develop mechanical allodynia, thereby showing that the set of peptides of the invention can be used preventively in a paw incision model (postoperative pain model).

### Example 5: Antalgic Effect in an Inflammatory Pain Model

The purpose of this experiment was to assess the antalgic effect of the set of peptides of SEQ ID NO: 1 and of SEQ ID NO:2 (“Peptides 1+2”) administered subcutaneously in vivo in an inflammatory pain model (capsaicin inflammatory pain model).

The experiments wee conducted on eight-week-old male WT C57Bl6. Two groups of 8 mice were used. “Peptides 1+2” is resuspended in HMPC solution at concentrations of 30 μg/mL, for a 10 μl injection per gram.

Capsaicin (Sigma) stock solution (dissolved in absolute ethanol 16.7 mg/mL) was diluted in NaCl 0.9% at 0.25 mg/mL, then 10 μL were injected into the plantar surface of the left hind paw of the mouse using a Hamilton syringe and a 26G needle. As previously described, measurement with Von Frey filaments by the up/down method for determining the baseline was done. 24 h after the injection of capsaicin (D+1), two groups of mice were treated with “Peptides 1+2” or a vehicle, respectively. The response threshold was measured at 1 hour (+1 h), 2 hours (+2 h) and then 4 hours (+4 h) followed by subcutaneous administration of “Peptides 1+2” solution at 0.3 mg/kg (n=8), or a vehicle solution (n=8).

24 h after injection of capsaicin, the mice developed mechanical allodynia (see at D+1—FIG. 7), while subcutaneous administration of the set of peptides of the invention induced a statistically significant increase in the response threshold two hours after administration.

These results show that the set of peptides of the invention caused an antalgic effect via subcutaneous administration in the capsaicin model (inflammatory pain model).

### Example 6: Antalgic Effect in an Osteoarthritis Pain Model

The purpose of this experiment was to assess the antalgic effect of the set of peptides of SEQ ID NO: 1 and of SEQ ID NO: 2 (“Peptides 1+2”) administered subcutaneously in vivo in osteoarthritis pain model (mono-iodoacetate model).

Two groups of 8 mice were used. Mechanical sensitivity was tested, using Von Frey filaments with the up/down method, before (Baseline) and 14 days after mono-iodoacetate (MIA) injection in each group. 14 days after the injection of MIA, two groups of mice were treated with “Peptides 1+2” or a vehicle (a solution of NaCl), respectively. A time course response was performed to follow antalgic effect of “Peptides 1+2”. The response threshold was measured 1 hour (+1 h), 2 hours (+2 h) and then 4 hours (+4 h) after the administration of “Peptides 1+2” or the vehicle.

At day 14 post-MIA injection, all mice exhibited a drastic decrease in the mechanical thresholds, illustrative of a strong mechanical hypersensitivity as compared to the baseline threshold (FIG. 8). Subcutaneous administration of the vehicle had no antalgic effect. On the contrary, at one hour and two hours post administration, the antalgic effect of “Peptide 1+2” was statistically significant in comparison with negative control (vehicle) (FIG. 8).

These results show that the set of peptides of the invention caused an antalgic effect via subcutaneous administration in an arthritis pain model.

### Example 7: Antalgic Effect in a Freeze-Burn Pain Model

The purpose of this experiment was to assess the antalgic effect of the set of peptides of SEQ ID NO: 1 and of SEQ ID NO: 2 (“Peptides 1+2”) administered subcutaneously in vivo in Freeze-burn pain model.

Two groups of 16 mice were used. Each group has 8 males and 8 females. Mechanical sensitivity was tested using Von Frey filaments with the up/down method before (Baseline) and at 24 h (D+1) after cold exposure. After the administration of “Peptides 1+2” or a vehicle (a NaCl solution) at 24 h after cold exposure, a time course response was performed to follow antalgic effect of “Peptides 1+2” one hour (D1+1), two hours (D1+2 h) and four hours (D1+4 h) post-administration.

24 hours after cold exposure, all mice exhibited a drastic decrease in the mechanical thresholds, illustrative of a strong mechanical hypersensitivity as compared to the baseline threshold (FIG. 9). Subcutaneous administration of the vehicle had no antalgic effect. On the contrary, at one hour and two hours post administration, the antalgic effect of “Peptide 1+2” was statistically significant in comparison with negative control (vehicle) (FIG. 9).

These results show that the set of peptides of the invention caused an antalgic effect via subcutaneous administration in a freeze burn (or ice burn) pain model. Such model is also considered as a type of acute pain model.

### Example 8: Antalgic Effect in a Ultraviolet Burn Pain Model

The purpose of this experiment was to assess the antalgic effect of the set of peptides of SEQ ID NO: 1 and of SEQ ID NO:2 (“Peptides 1+2”) administered subcutaneously in vivo in Ultraviolet-burn pain model (also called sun burn pain model).

Two groups of 16 mice were used. Each group has 8 males and 8 females. Mechanical sensitivity was then tested using Von Frey filaments with the up/down method before (Baseline) and at 24 h (D+1) after ultraviolet irradiation. After the administration of “Peptides 1+2” or a vehicle (a NaCl solution), a time course response was performed to follow antalgic effect of “Peptides 1+2” one hour (D1+1 h), two hours (D1+2 h) and four hours (D1+4 h) post-administration.

24 hours after ultraviolet irradiation, all mice exhibited a drastic decrease in the mechanical thresholds, illustrative of a strong mechanical hypersensitivity as compared to the baseline threshold (FIG. 10). Subcutaneous administration of the vehicle had no antalgic effect. On the contrary, at one hour and two hours post administration, the antalgic effect of the set “Peptide 1+2” was statistically significant in comparison with negative control (vehicle) (FIG. 10).

These results show that the set of peptides of the invention caused an antalgic effect via subcutaneous administration in an UV burn (or sun burn) pain model. Such model is also considered as a type of acute pain model.

### REFERENCES

Benyamin, R., Trescot, A. M., Datta, S., Buenaventura, R., Adlaka, R., Sehgal, N., Glaser, S. E., and Vallejo, R. (2008). Opioid complications and side effects. Pain Physician 11, S105-120.
Bourane, S., Duan, B., Koch, S. C., Dalet, A., Britz, O., Garcia-Campmany, L., Kim, E., Cheng, L., Ghosh, A., Ma, Q., et al. (2015a). Gate control of mechanical itch by a subpopulation of spinal cord interneurons. Science 350, 550-554.
Bourane, S., Grossmann, K. S., Britz, O., Dalet, A., Del Barrio, M. G., Stam, F. J., Garcia-Campmany, L., Koch, S., and Goulding, M. (2015b). Identification of a spinal circuit for light touch and fine motor control. Cell 160, 503-515.
Boyle, K. A., Gradwell, M. A., Yasaka, T., Dickie, A. C., Polgar, E., Ganley, R. P., Orr, D. P. H., Watanabe, M., Abraira, V. E., Kuehn, E. D., et al. (2019). Defining a Spinal Microcircuit that Gates Myelinated Afferent Input: Implications for Tactile Allodynia. Cell reports 28, 526-540 e526.
Colloca, L., Ludman, T., Bouhassira, D., Baron, R., Dickenson, A. H., Yarnitsky, D., Freeman, R., Truini, A., Attal, N., Finnerup, N. B., et al. (2017). Neuropathic pain. Nat Rev Dis Primers 3, 17002.
Costigan, M., Scholz, J., and Woolf, C. J. (2009). Neuropathic pain: a maladaptive response of the nervous system to damage. Annu Rev Neurosci 32, 1-32.
Coull, J. A., Beggs, S., Boudreau, D., Boivin, D., Tsuda, M., Inoue, K., Gravel, C., Salter, M. W., and De Koninck, Y. (2005). BDNF from microglia causes the shift in neuronal anion gradient underlying neuropathic pain. Nature 438, 1017-1021.
Delfini, M. C., Mantilleri, A., Gaillard, S., Hao, J., Reynders, A., Malapert, P., Alonso, S., Francois, A., Barrere, C., Seal, R., et al. (2013). TAFA4, a chemokine-like protein, modulates injury-induced mechanical and chemical pain hypersensitivity in mice. Cell reports 5, 378-388.
Duan, B., Cheng, L., Bourane, S., Britz, O., Padilla, C., Garcia-Campmany, L., Krashes, M., Knowlton, W., Velasquez, T., Ren, X., et al. (2014). Identification of spinal circuits transmitting and gating mechanical pain. Cell 159, 1417-1432.
Goh, E L., Chidambaram, S., Ma, D. (2017). Complex regional pain syndrome: a recent update. Burns & Trauma 5:2.
Leroux, Maria Bibiana (2018). Erythromelalgia: a cutaneous manifestation of neuropathy? An Bras Dermatol. 93(1):86-94.
Ngo, A L., Urits, I., Yilmaz, M., Fortier, L., Anya, A., Oh, J H., Berger, A A., Kassem, H., Sanchez, MG., Kaye, A D, et al. (2020). Postherpetic Neuralgia: Current Evidence on the Topical Film-Forming Spray with Bupivacaine Hydrochloride and a Review of Available Treatment Strategies. Adv Ther. 37(5): 2003-2016.
Peirs, C., Williams, S. P., Zhao, X., Walsh, C. E., Gedeon, J. Y., Cagle, N. E., Goldring, A. C., Hioki, H., Liu, Z., Marell, P. S., et al. (2015). Dorsal Horn Circuits for Persistent Mechanical Pain. Neuron 87, 797-812.
Petitjean, H., F, B. B., Tsao, D., Davidova, A., Sotocinal, S. G., Mogil, J. S., Kania, A., and Sharif-Naeini, R. (2019). Recruitment of Spinoparabrachial Neurons by Dorsal Horn Calretinin Neurons. Cell reports 28, 1429-1438 e1424.
Petitjean, H., Pawlowski, S. A., Fraine, S. L., Sharif, B., Hamad, D., Fatima, T., Berg, J., Brown, C. M., Jan, L. Y., Ribeiro-da-Silva, A., et al. (2015). Dorsal Horn Parvalbumin Neurons Are Gate-Keepers of Touch-Evoked Pain after Nerve Injury. Cell reports 13, 1246-1257.
Sarver, D. C., Lei, X., and Wong, G. W. (2021). FAM19A (TAFA): An Emerging Family of Neurokines with Diverse Functions in the Central and Peripheral Nervous System. ACS Chem Neurosci 12, 945-958.
Siracusa, R., Di Paola, R., Cuzzocrea. S., Impellizzeri, D. (2021). Fibromyalgia: Pathogenesis, Mechanisms, Diagnosis and Treatment Options Update. Int J Mol Sci. 22(8): 3891.
Tom Tang, Y., Emtage, P., Funk, W. D., Hu, T., Arterburn, M., Park, E. E., and Rupp, F. (2004). TAFA: a novel secreted family with conserved cysteine residues and restricted expression in the brain. Genomics 83, 727-734.
Zeilhofer, H. U., Wildner, H., and Yevenes, G. E. (2012). Fast synaptic inhibition in spinal sensory processing and pain control. Physiol Rev 92, 193-235.
Zhang, Y., Liu, S., Zhang, Y. Q. Goulding, M., Wang, Y. Q., and Ma, Q. (2018). Timing Mechanisms Underlying Gate Control by Feedforward Inhibition. Neuron 99, 941-955 e944.

1-16. (canceled)

17. A composition comprising a first peptide set forth in SEQ ID NO: 1 or a peptide having at least 90% identity to SEQ ID NO: 1, and a second peptide set forth in SEQ ID NO: 2 or a peptide having at least 90% identity to SEQ ID NO: 2, wherein said two peptides are not linked to each other.

18. The composition of claim 17, wherein if the composition comprises a peptide having at least 90% identity to SEQ ID NO: 1 and/or a peptide having at least 90% identity to SEQ ID NO: 2, the glutamine (Q) residue at position 13 in the peptide of SEQ ID NO: 1 remains unchanged in the amino acid sequence of the peptide having at least 90% identity to SEQ ID NO: 1, and the tyrosine (Y) residue at position 18 in the peptide of SEQ ID NO: 2 either remains unchanged in the amino acid sequence of the peptide having at least 90% identity to SEQ ID NO: 2 or is replaced by a serine (S).

19. The composition of claim 17, said composition further comprising at least one additional active compound.

20. The composition of claim 19, wherein the at least one additional active compound is a SAID, NSAID or opioid drug.

21. The composition of claim 17, wherein the composition comprises a pharmaceutically and/or dietetically acceptable support.

22. The composition of claim 17, wherein the composition further comprises ascorbic acid or an ascorbic acid salt.

23. A set of components, or composition, comprising nucleic acids allowing the expression of two peptides which are not linked to each other, wherein a first nucleic acid comprises a sequence encoding a peptide set forth in SEQ ID NO: 1 or a peptide having at least 90% identity to SEQ ID NO: 1, and a second nucleic acid comprises a sequence encoding a peptide set forth in SEQ ID NO: 2, or a peptide having at least 90% identity to SEQ ID NO: 2.

24. The set of components, or composition, of claim 23, said nucleic acids allowing expression of two peptides which are not linked to each other comprising vectors, wherein a first vector comprises a sequence encoding a peptide set forth in SEQ ID NO: 1 or a peptide having at least 90% identity to SEQ ID NO: 1, and a second vector comprises a sequence encoding a peptide set forth in SEQ ID NO: 2, or a peptide having at least 90% identity to SEQ ID NO: 2.

25. A cell or composition comprising said cell and an acceptable support, wherein the cell comprises a set of components comprising nucleic acids of claim 23.

26. A method of preventing or treating pain in a subject comprising administering a composition of claim 17 to a subject in need of pain treatment.

27. The method of claim 26, wherein said pain is a chronic pain, a neuropathic pain, a postoperative pain, an inflammatory pain, an arthritic pain, pain associated with damage to joints, pain related to Ehlers-Danlos syndrome, pain resulting from sun burn, pain resulting from ice burn, hyperalgesia, allodynia, or acute pain.

28. The method of claim 26, wherein the subject is a non-human mammal or a human being.

29. The method of claim 26, wherein the composition is administered intramuscularly, intravenously, intraperitoneally, orally (per os), anally, cutaneously, subcutaneously, topically, dermically, transdermically or intrathecally to the subject.

30. The method of claim 29, wherein the composition is administered subcutaneously or orally.

31. The method of claim 26, wherein said composition comprises between 1 μg and 100 mg of both the first and second peptides per unit dose.

32. The method of claim 26, wherein said composition is administered at a dose between 1 μg/kg/day and 100 mg/kg/day.

33. The method of claim 26, wherein the composition is administered at a dose between 2.5 μg/kg/day and 0.6 mg/kg/day for a mammal.

34. A kit comprising i) a first peptide of SEQ ID NO: 1 or a peptide having at least 90% identity to SEQ ID NO: 1, or a nucleic acid sequence encoding said peptide, ii) a second peptide of SEQ ID NO: 2 or a peptide having at least 90% identity to SEQ ID NO: 2, or a nucleic acid sequence encoding said peptide, the first and second peptides, or the nucleic acids encoding said peptides, being in distinct containers, and optionally iii) written instructions for using the kit.

35. A method of preventing or treating pain in a subject comprising administering a set of components or a composition according to claim 23 to a subject in need of pain treatment.

36. A method of preventing or treating pain in a subject comprising administering a cell or composition according to claim 25 to a subject in need of pain treatment.


SEQUENCE LISTING (6 sequences)

SEQ ID NO: 1 - protein, 27 aa, synthetic construct
  REGION 1..27: note = Peptide 1
CFPGQVAGTTRAQPSCVEASIVIQKWW

SEQ ID NO: 2 - protein, 36 aa, synthetic construct
  REGION 1..36: note = Peptide 2
CHMNPCLEGEDCKVLPDYSGWSCSSGNKVKTTKVTR

SEQ ID NO: 3 - DNA, 81 bp, synthetic construct
  misc_feature 1..81: note = Peptide 1's coding sequence
  source 1..81: mol_type = other DNA
tgcttcccgggacaggtggcgggcacaactcgggctcaaccttcttgtgttgaagcttccattgtgattcagaaatggtgg

SEQ ID NO: 4 - DNA, 108 bp, synthetic construct
  misc_feature 1..108: note = Peptide 2's coding sequence
  source 1..108: mol_type = other DNA
tgtcacatgaatccgtgtttggaaggagaggattgtaaagtgctgccagattactcaggttggtcctgtagcagtggcaataaagtcaaaactacgaaggtaacgcgg

SEQ ID NO: 5 - protein, 105 aa, synthetic construct
  REGION 1..105: note = Mature human TAFA-4 protein
SQHLRGHAGHHQIKQGTCEVVAVHRCCNKNRIEERSQTVKCSCFPGQVAGTTRAQPSCVEASIVIQKWWCHMNPCLEGEDCKVLPDYSGWSCSSGNKVKTTKVTR

SEQ ID NO: 6 - protein, 36 aa, synthetic construct
  REGION 1..36: note = Peptide 2 (18-S)
CHMNPCLEGEDCKVLPDSSGWSCSSGNKVKTTKVTR